(19) United States
(12) Patent Application Publication
(10) Pub. No.: US 2016/0310410 A1
(43) Pub. Date: Oct. 27, 2016

(54) PHARMACEUTICAL COMPOSITIONS COMPRISING ARFORMOTEROL AND GLYCOPYRRONIUM

(71) Applicant: GLENMARK SPECIALTY S.A., La Chaux-de-Fonds (CH)

(72) Inventors: Ulhas Dhuppad, Nashik (IN); Franciscus KOPPENHAGEN, Deerfield Beach, FL (US); Ashok KATKURWAR, Nashik (IN); Ramakant CHANAGARE, Nashik (IN); Kautik SHIROLE, Nashik (IN); Jitendra PATIL, Nashik (IN); Rajesh ANKAM, Nashik (IN); Nainesh PATEL, Nashik (IN)

(21) Appl. No.: 15/137,494

(22) Filed: Apr. 25, 2016

(30) Foreign Application Priority Data
Apr. 24, 2015 (IN) 1657/MUM/2015
Jan. 21, 2016 (IN) 201621002297

(51) Int. Cl. A61K 9/00; A61K 47/06; A61K 47/10; A61K 47/32; A61K 31/167; A61K 31/40

(52) U.S. Cl. CPC A61K 9/008; A61K 31/167; A61K 31/40; A61K 47/10; A61K 47/32; A61K 47/06

(57) ABSTRACT

Described herein is a fixed dose pharmaceutical composition containing arformoterol or its salt and a glycopyrronium salt. Particularly, a fixed dose pharmaceutical composition in the form of an aerosol or an aerosolizable for inhalation administration includes arformoterol or its salt, a glycopyrronium salt, and pharmaceutically acceptable excipients. Also described is a process for preparing the compositions and their use for the treatment of asthma and/or chronic obstructive pulmonary disease in a subject in need thereof. Also described are pharmaceutical compositions that include an effective amount of arformoterol (or its salt), a glycopyrronium salt or their combination in the form of a suspension or solution, and methods of their preparation.

### CROSS REFERENCE TO RELATED APPLICATION

This application claims priority of Indian Patent Application Nos. 1657/MUM/2015 filed on Apr. 24, 2015 and IN 201621002297 filed on Jan. 21, 2016, which are hereby incorporated by reference in their entirety.

### TECHNICAL FIELD

Described herein is a fixed dose pharmaceutical composition comprising arformoterol or its salt and a glycopyrronium salt. Particularly, disclosed herein is a fixed dose pharmaceutical composition in the form of an aerosol for inhalation administration comprising arformoterol or its salt, a glycopyrronium salt, and pharmaceutically acceptable excipients. Also described is a process for preparing such a composition and its use for the treatment of asthma and/or chronic obstructive pulmonary disease in a subject in need thereof. More specifically, a pharmaceutical composition comprises an effective amount of arformoterol (or its salt), glycopyrronium salt or its combination in the form of a suspension or solution, and a method of its preparation.

### BACKGROUND

Respiratory disorders related to airway inflammation include a number of lung diseases such as chronic obstructive pulmonary disease (COPD) and asthma.

Asthma is characterized by an increased responsiveness of the trachea and bronchi to various stimuli, and is manifested by a widespread narrowing of the airways that changes in severity either spontaneously or as a result of treatment. The events leading to airway obstruction in asthma include edema of airway walls, infiltration of inflammatory cells into the lung, production of various inflammatory mediators, and increased mucous production.

The current therapy for asthma includes bronchodilator drugs, corticosteroids and leukotriene antagonists. Bronchodilator drugs dilate the bronchi and bronchioles, decrease resistance in the respiratory airway, and increase airflow to the lungs. Corticosteroid drugs are effective at reducing asthma symptoms by blocking the body's inflammatory response. The leukotriene antagonists have limited efficacy, producing only small increase in pulmonary function as demonstrated in clinical trials.

COPD is a term used to classify two major airflow obstruction disorders: chronic bronchitis and emphysema. Chronic bronchitis is inflammation of the bronchial airways. Emphysema is an over inflation of the alveoli, or air sacs in the lungs. Emphysema has a number of causes, including smoking, exposure to environmental pollutants, alpha-one antitrypsin deficiency, and aging. COPD is a disease of the respiratory apparatus, characterized by an irreversible obstruction of the airways, of a degree that varies according to the gravity.

There are very limited therapies currently available to arrest COPD progression and otherwise prevent its exacerbation, preserve lung function, and otherwise improve the quality of life of COPD patients. The arsenal of medications available to practitioners treating COPD patients have traditionally included: fast-acting β2-agonists, anticholinergic bronchodilators, long-acting bronchodilators, antibiotics, and expectorants. The currently available treatments for COPD exhibit short term benefits, however no long term effects were found on COPD progression following administration of anti-cholinergic drugs, adrenergic agonists, and oral steroids.

Arformoterol (N-[2-hydroxy-5-[(1R)-1-hydroxy-2-[[(2R)-1-(4-methoxyphenyl) propan-2-yl] amino] ethyl] phenyl] formamide.) is a long acting beta-adrenoceptor agonist. It is commercially available in the U.S. as Brovana®, in the form of an inhalation solution, and is administered twice daily (morning and evening) by nebulization. It is indicated for the treatment of chronic obstructive pulmonary disease (COPD).

Glycopyrronium (3-[(cyclopentylhydroxyphenylacetyl)oxy]-1,1-dimethyl pyrrolidinium) is an anti-muscarinic agent. It is commercially available in the U.S. as a dry powder inhaler, tablets and in an injectable form.

WO 2010/138862, WO 2010/138868, WO 2010/138884 and WO2012/158166 disclose compositions that include a suspension medium, active agent particles and suspending particles, in which the active agent particles and suspending particles form a co-suspension within the suspension medium.

WO 2011/076843 discloses an aerosol formulation suitable for administering to COPD patients by means of a pressurized metered dose inhaler (pMDI), comprising glycopyrronium bromide in combination with formoterol in solution form.

WO 2013/021199 discloses a pharmaceutical composition comprising a eutectic composition of two pharmacologically active ingredients for delivery to the lung by inhalation.

WO 2001/076575 discloses a controlled release formulation for pulmonary delivery of glycopyrrolate.

WO 2005/074918 discloses a combination of an anticholinergic component and a glucocorticoid component, and their use for treating diseases of the respiratory tract.

US Patent Publication No. 20050118107 discloses a pMDI formulation containing formoterol and a blend of propellants for use in the treatment of inflammatory conditions/disorders, especially respiratory diseases such as asthma, COPD and rhinitis.

US Patent Publication No. 20130142879 discloses suspension formulations, especially those for delivering a pharmaceutically active agent in aerosol form using a spray or aerosol device, such as a pressurized metered dose inhaler.

U.S. Pat. No. 8,518,377 discloses pressurized gas formulations for dosage aerosols, in which a medicament is formulated suspended in TG 227 ea (HFA 227, 1,1,1,2,3,3,3-heptafluoropropane) and/or TG 134a (HFA 134a, 1,1,1,2-tetrafluoroethane) as a propellant, and to their use for producing a medicament.

U.S. Pat. No. 6,713,047 discloses an aerosol inhaler which includes an active material, a propellant containing a hydrofluoroalkane, a cosolvent, and optionally a low volatility compound. The use of a mixture of HFA 134a (1,1,1,2-tetrafluoroethane) and HFA 227 (1,1,1,2,3,3,3-heptafluoropropane) allows one to modulate the mass median aerodynamic diameter (MMAD) of the aerosol particles on actuation of the inhaler to target specific regions of the respiratory tract.

US Patent Publication No. 20070286814 discloses stable aerosol pharmaceutical formulation of albuterol sulfate, ipratropium bromide, or a combination thereof, in combination with a cosolvent and optionally a surfactant.

There still exists a need for an effective therapeutic treatment for respiratory diseases such as asthma and COPD. This disclosure fulfills this need and provides additional advantages described herein.

### SUMMARY

Described herein is a fixed dose pharmaceutical composition in the form of an aerosol or an aerosolizable composition for inhalation administration comprising an effective amount of arformoterol or its salt and an effective amount of a glycopyrronium salt.

In an embodiment, a fixed dose pharmaceutical composition (e.g., a suspension) in the form of an aerosol or an aerosolizable composition for inhalation administration comprises (a) about 0.0005% w/w to about 0.05% w/w of arformoterol or its salt, (b) about 0.005% w/w to about 0.05% w/w of a glycopyrronium salt, and (c) a pharmaceutically acceptable excipient selected from lubricants, surfactants, co-solvents, stabilizing agents, dispersing agents, bulking agents, buffers, complexing agents, preservatives, osmotic agents, and combinations thereof.

In another embodiment, a fixed dose pharmaceutical suspension in the form of an aerosol or an aerosolizable composition for inhalation administration comprises (a) about 0.0005% w/w to about 0.05% w/w of arformoterol or its salt (b) about 0.005% w/w to about 0.05% w/w of a glycopyrronium salt (c) about 0.02% w/w of polyethylene glycol 1000, and (d) about 0.0001% w/w of polyvinyl pyrrolidone and (e) a propellant selected from HFA 134a, HFA 227, and a mixture of HFA 227 and HFA 134a, all weights based on the total weight of the composition.

In another embodiment, a pharmaceutical composition in the form of an aerosol or an aerosolizable composition for inhalation administration contains arformoterol as the only active agent. In this embodiment the pharmaceutical composition comprises an active agent and one or more pharmaceutically acceptable excipients, the active agent consisting of about 0.0005% w/w to about 0.05% w/w of arformoterol or its salt.

In another embodiment, a pharmaceutical composition in the form of an aerosol or an aerosolizable composition for inhalation administration comprises an active agent and one or more pharmaceutically acceptable excipients, the active agent consisting of about 0.1 μg to about 7.5 μg per actuation of arformoterol or its salt.

In another embodiment, a pharmaceutical composition in the form of an aerosol or an aerosolizable composition for inhalation administration consists essentially of about 0.0005% w/w to about 0.05% w/w of arformoterol or its salt, 0.00001% w/w to about 0.002%, w/w of polyvinyl pyrrolidone, 0.005% w/w to about 0.08% w/w of polyethylene glycol, and a propellant selected from HFA 134a, HFA 227, and a mixture of HFA 227 and HFA 134a, all weights based on the total weight of the composition.

In another embodiment, a pharmaceutical composition in the form of an aerosol or an aerosolizable composition for inhalation administration comprises an active agent and one or more pharmaceutically acceptable excipients, the active agent consisting of about 0.005% w/w to about 0.05% w/w of a glycopyrronium salt.

In another embodiment, a pharmaceutical composition in the form of an aerosol or an aerosolizable composition for inhalation administration comprises an active agent and one or more pharmaceutically acceptable excipients, the active agent consisting of about 1 μg to about 30 μg per actuation of a glycopyrronium salt.

In another embodiment, a pharmaceutical composition in the form of an aerosol or an aerosolizable composition for inhalation administration consists essentially of about 0.005% w/w to about 0.05% w/w of a glycopyrronium salt, about 0.00001% w/w to about 0.0.002%, w/w of polyvinyl pyrrolidone, about 0.005% w/w to about 0.08% w/w of polyethylene glycol, and a propellant selected from HFA 134a, HFA 227, and a mixture of HFA 227 and HFA 134a, all weights based on the total weight of the composition.

### DETAILED DESCRIPTION

Described herein are fixed dose pharmaceutical compositions in the form of an aerosol or an aerosolizable composition for inhalation administration, methods of making the compositions, and methods of treating subjects, such as human subjects. In some aspects, the compositions comprise an effective amount of arformoterol or its salt and an effective amount of a glycopyrronium salt. Compositions comprising arformoterol or its salt as the only active agent and compositions comprising a glycopyrronium salt as the only active agent are also provided. In one embodiment, the pharmaceutical composition is a stable suspension in the form of an aerosol of arformoterol (or its salt) and a glycopyrronium salt. The inventors discovered that a stable suspension of arformoterol or its salt and a glycopyrronium salt could be formed that can produce an aerosol with a sufficiently small mass median aerodynamic diameter upon actuation (e.g., less than 5 microns) to ensure deposition of the active ingredients below the larynx upon inhalation and a uniformity of delivered dose which provides repeatable and predictable dosing. Also disclosed herein are pharmaceutical compositions comprising an effective amount of arformoterol (or its salt) and/or a glycopyrronium salt in the form of a suspension or solution, and a methods of their preparation.

The pharmaceutical compositions described herein may have one or more, or all, of the following characteristics: (i) can be in the form of an aerosol, such as actuation of a solution or suspension from a metered dose inhaler (MDI), having a mass median aerodynamic diameter of less than 5 microns, specifically less than 4 microns, (ii) does not undergo phase separation when stored at 25±2° C. and 60%±5% relative humidity for at least 30 minutes, and/or (iii) has a good uniformity of delivered aerosol dose (as measured by the initial, middle and end of the aerosol form). Preferably, neither of the active ingredients in the composition readily crystallizes during storage.

More particularly, it has been found that stable fixed dose compositions in the form of an aerosol or an aerosolizable composition for inhalation administration containing arformoterol or its salt (e.g., arformoterol tartrate) and/or a glycopyrronium salt (e.g., a glycopyrronium bromide) can be produced. In one advantageous aspect, the stable compositions may include less than 0.002% w/w, or less than 0.0015% w/w, or less than 0.001% w/w of polyvinyl pyrrolidone, which is much lower than the amounts typically used in aerosol compositions. In another advantageous aspect, the total weight percentage of arformoterol or its salt, glycopyrronium salt, or a combination thereof, is less than or equal to the total weight percentage of soluble excipients, e.g., polyethylene glycol. In yet another advantageous aspect, the total weight percentage of arformoterol or its salt, glycopyrronium salt, or a combination thereof, is greater than or equal to the total weight percentage of insoluble excipients, e.g., polyvinyl pyrrolidone.

It has also unexpectedly been found that a lower dose of arformoterol or its salt than has been used previously can be employed to provide an efficacious aerosol composition for inhalation administration.

In one embodiment, a fixed dose pharmaceutical composition in the form of an aerosol or an aerosolizable composition for inhalation administration comprises an effective amount of arformoterol or its salt and an effective amount of a glycopyrronium salt.

In one aspect, the composition comprises about 0.00001% w/w to about 1% w/w, about 0.0001% w/w to about 0.1% w/w, about 0.0005% w/w to about 0.05% w/w of arformoterol or its salt, and about 0.00001% w/w to about 1% w/w, about 0.0001% w/w to about 0.1% w/w, or about 0.005% w/w to about 0.05% w/w of a glycopyrronium salt, based upon the total weight of the composition.

In any of the aspects included herein, the effective amount of arformoterol salt is equivalent to about 0.001 μg to about 50 μg of arformoterol, or about 0.01 μg to about 30 μg of arformoterol, or about 0.1 μg to about 10 μg of arformoterol, or about 0.1 μg to about 7.5 μg of arformoterol, or about 6.0 μg of arformoterol, or about 5 μg of arformoterol, or about 4.0 μg of arformoterol per actuation. Exemplary effective amounts per actuation of arformoterol salt are equivalent to 0.5 μg, 0.75 μg, 1 μg, 1.5 μg and 3 μg of arformoterol.

The effective amount of the glycopyrronium salt can be about 0.01 μg to about 80 μg per actuation, about 0.01 μg to about 40 μg per actuation, or about 1 μg to about 30 μg per actuation. Weights given for glycopyrronium salt are based on the weight of glycopyrronium bromide, the weights for other salt forms differs slightly. For example 30 μg glycopyrronium salt, based on the weight of glycopyrronium bromide, is equivalent to 26.6 μg glycopyrronium chloride. Exemplary effective amounts per actuation of glycopyrronium salt are 3.75 μg, 7.5 μg and 11.25 μg.

In any of the aspects included herein, the arformoterol or its salt and the glycopyrronium salt are present in a weight ratio of arformoterol or its salt to glycopyrronium salt of about 1:0.001 to about 1:100, about 1:0.001 to about 1:70, about 1:0.005 to 1:50, about 1:0.05 to about 1:40, or about 1:0.70 to about 1:30.

In any of the aspects described herein, the molar ratio of arformoterol or its salt to glycopyrronium salt is about 1:0.0001 to about 1:200, about 1:0.001 to about 1:150, about 1:0.01 to 1:100, about 1:0.1 to about 1:50, or about 1:1 to about 1:30.

In any of the aspects described herein, the concentration of arformoterol or its salt in the composition (e.g., suspension) is about 0.0001 mg/ml to about 10 mg/ml, about 0.001 mg/ml to 1 mg/ml, about 0.01 mg/ml to about 0.5 mg/ml, or about 0.026 mg/ml to about 0.1 mg/ml.

In any of the aspects described herein, the concentration of glycopyrronium salt in the composition (e.g., suspension) is about 0.001 mg/ml to about 10 mg/ml, about 0.01 mg/ml to 1 mg/ml, about 0.05 mg/ml to about 0.5 mg/ml, or about 0.09 mg/ml to about 0.3 mg/ml.

In any of the aspects included herein, the arformoterol or its salt and/or the glycopyrronium salt are present in suspended particulate form in the composition. For example, the active agent can be in suspended form due to low solubility.

In any of the compositions described herein, the arformoterol or its salt (e.g., arformoterol tartrate) and/or the glycopyrronium salt (e.g., glycopyrronium bromide) has a mean particle size (D₅₀) in suspension of about 1 μm to about 10 μm, or in some embodiments a particle has D₉₀ of below 6 μm.

The aerosols or aerosolizable compositions can further comprise a propellant. Thus, in an embodiment, a fixed dose pharmaceutical composition in the form of an aerosol or an aerosolizable composition for inhalation administration comprises (a) an effective amount of arformoterol or its salt, (b) an effective amount of a glycopyrronium salt, (c) a propellant, selected from HFA 134a, HFA 227, or a mixture of HFA 227 and HFA 134a, and (d) optionally one or more pharmaceutically acceptable excipients. In one aspect, the composition comprises at least about 30% w/w of the HFA 227 in the mixture of HFA 227 and HFA 134a, based upon the weight of the propellant. In one aspect, the composition comprises at least about 30% w/w of the HFA 134a in the mixture of HFA 227 and HFA 134a, based upon the weight of the propellant.

In some embodiments, the ratio of HFA 227:HFA 134a is about 0:100 to about 100:0, about 20:80 to about 80:20, about 40:60 to about 60:40, or about 30:70 to about 70:30. In other embodiments, the composition is substantially free of HFA 134a.

The fixed dose compositions can include soluble pharmaceutically acceptable excipients, and/or insoluble pharmaceutically acceptable excipients.

In one aspect, fixed dose pharmaceutical composition in the form of an aerosol an aerosolizable composition for inhalation administration comprises (a) arformoterol or its salt, (b) a glycopyrronium salt, (c) a polymer, and (d) one or more pharmaceutically acceptable excipients.

In one aspect, fixed dose pharmaceutical composition in the form of an aerosol or an aerosolizable composition for inhalation administration comprises (a) arformoterol or its salt, (b) a glycopyrronium salt, (c) a stabilizing amount of surfactant, and (d) one or more pharmaceutically acceptable excipients.

In one aspect, the total weight of arformoterol or its salt and/or the glycopyrronium salt may be equal to or greater than the total weight of pharmaceutically acceptable excipients. In another aspect, the total weight of arformoterol or its salt and/or the glycopyrronium salt is equal to or less than the total weight of soluble pharmaceutically acceptable excipients. In another aspect, the weight of arformoterol or its salt and/or glycopyrronium salt, is equal to or greater than the total weight of pharmaceutically acceptable excipients present in substantially insoluble form. In one aspect, the total weight of arformoterol or its salt and the glycopyrronium salt is equal to or less than the total weight of soluble pharmaceutically acceptable excipients.

In one aspect, a stable suspension in the form of an aerosol or an aerosolizable composition for inhalation administration to a human comprises (a) arformoterol or its salt in an amount of about 0.1 μs to about 7.5 μg, (b) a glycopyrronium salt in the amount of about 1 μg to about 30 μg, (c) about 0.0001% w/w of excipient present in substantially insoluble form, (d) about 0.02% w/w of excipient present in soluble form, and (e) at least one propellant.

Exemplary soluble and insoluble pharmaceutically acceptable excipients include lubricants, surfactants, co-solvents, stabilizing agents, dispersing agents, bulking agents, buffers, complexing agents, preservatives, osmotic agents, and combinations thereof.

Polyethylene glycol (PEG) or its derivatives such as polyethylene glycol are specific examples of a soluble pharmaceutically acceptable excipient, which can act as a lubricant in the compositions. PEG derivatives include —(CH₂CH₂O)ₙ-recurring units, wherein n is an integer ≧2. In certain embodiments n is ≧4, ≧6 or ≧8. In one embodiment, n is ≦20. Preferred PEG derivatives are linear. Most preferably, polyethylene glycol (PEG), i.e. HO—(CH2CH2O)ₙ—H. Preferably the average molecular weight of the PEG or PEG derivative is 50 to 6000 Da, 100 to 5000 Da, or 200 to 4000 Da. PEGs include PEG 1000, which is PEG having a molecular weight of 1000.

In a specific embodiment, the one or more substantially insoluble pharmaceutically acceptable excipients is polyvinyl pyrrolidone (povidone), which can act as a surfactant in the compositions. Different types of PVP may be characterized by their viscosity in solution, expressed as a K-value. In certain embodiments the K-value of the PVP used is between 10 and 150, between 15 and 80, between 20 and 40, or about 30. Suitable polyvinyl pyrrolidones are PVP (K30), PVP (K25), PVP (K30), PVP (K29/32), PVP (K32), PVP (K90), PVP (K120), PVP (C15), PVP (C30) or PVP/17PF.

In one aspect, a fixed dose pharmaceutical composition in the form of an aerosol or an aerosolizable composition for inhalation administration comprises an effective amount of arformoterol or its salt and an effective amount of a glycopyrronium salt, polyvinyl pyrrolidone and polyethylene glycol. In one aspect, the polyvinyl pyrrolidone is present in substantially insoluble form and the polyethylene glycol is present in dissolved form. The foregoing embodiments may include a propellant such as HFA 134a, HFA 227, or a mixture of HFA 227 and HFA 134a.

In another embodiment, a fixed dose pharmaceutical composition in the form of an aerosol or an aerosolizable composition for inhalation administration comprises (a) arformoterol or its salt, (b) a glycopyrronium salt, (c) about 0.005% w/w to about 0.08% w/w, or about 0.01% w/w to about 0.05% w/w of a polyethylene glycol, e.g., a polyethylene glycol having a molecular weight of 200 to 6000 (e.g., PEG 1000), and (d) about 0.00001% w/w to about 0.001% w/w, or about 0.00005% w/w to about 0.01% w/w of polyvinyl pyrrolidone, all based upon the total weight of the composition.

In a more specific embodiment, a fixed dose pharmaceutical composition in the form of an aerosol or an aerosolizable composition for inhalation administration comprises, consisting essentially of, or consists of (a) about 0.0005% w/w to about 0.05% w/w of arformoterol or its salt, (b) about 0.005% w/w to about 0.05% w/w of a glycopyrronium salt, (c) about 0.02% w/w of polyethylene glycol 1000, (d) about 0.0001% w/w of polyvinyl pyrrolidone (e.g., PVP K25), and (e) propellant selected from HFA 134a, HFA 227, or a mixture of HFA 227 and HFA 134a, all based upon the total weight of the composition.

In another embodiment, a fixed dose pharmaceutical suspension comprising (a) an effective amount of arformoterol tartrate and an effective amount of a glycopyrronium bromide, (b) less than about 0.0015% w/w polyvinyl pyrrolidone (c) about 0.02% w/w polyethylene glycol and (d) a propellant selected from HFA 134a, HFA 227, or a mixture of HFA 227 and HFA 134a, all based upon the total weight of the composition, wherein the composition is in the form of an aerosol or an aerosolizable composition for inhalation administration

In one embodiment, included herein is a pharmaceutical composition in the form of an aerosol or an aerosolizable composition for inhalation administration, wherein the active agent consists of about 0.0005% w/w to about 0.05% w/w of arformoterol or its salt. The composition can include one or more pharmaceutically acceptable excipients.

In another embodiment, included herein is a pharmaceutical composition in the form of an aerosol or an aerosolizable composition for inhalation administration, wherein the active agent consists of about 0.1 μg to about 7.5 μg per actuation of arformoterol or its salt.

In certain aspects, the compositions wherein the active agent consists of arformoterol or its salt optionally include polyvinyl pyrrolidone, polyethylene glycol, a propellant or a combination thereof.

The propellant can be HFA 134a, HFA 227, or a mixture of HFA 227 and HFA 134a. In one aspect, the composition comprises at least about 30% w/w of the HFA 227 in the mixture of HFA 227 and HFA 134a, based upon the weight of the propellant. In one aspect, the composition comprises at least about 30% w/w of the HFA 134a in the mixture of HFA 227 and HFA 134a, based upon the weight of the propellant. In some embodiments, the ratio of HFA 227:HFA 134a is about 0:100 to about 100:0, about 20:80 to about 80:20, about 40:60 to about 60:40, or about 30:70 to about 70:30. In other embodiments, the composition is substantially free of HFA 134a.

The polyethylene glycol, when present, is present in an amount of about 0.005% w/w to about 0.08% w/w, or about 0.01% w/w to about 0.05% w/w, based upon the total weight of the composition. In a specific embodiment, the polyethylene glycol is present in an amount of 0.02% w/w and is polyethylene glycol 1000.

The polyvinyl pyrrolidone, when present, is present in an amount of about 0.00001% w/w to about 0.01% w/w, or about 0.00005% w/w to about 0.001% w/w, based upon the total weight of the composition. In one embodiment, the polyvinyl pyrrolidone is present in an amount of less than 0.0015% w/w. In a specific embodiment, the polyvinyl pyrrolidone is present in an amount of about 0.0001% w/w of polyvinyl pyrrolidone (e.g., PVP K25).

In one aspect, a pharmaceutical composition in the form of an aerosol or an aerosolizable composition for inhalation administration consists of about 0.0005% w/w to about 0.05% w/w of arformoterol or its salt, about 0.00001% w/w to about 0.01% w/w of polyvinyl pyrrolidone, about 0.005% w/w to about 0.08% w/w of polyethylene glycol, and a propellant selected from HFA 134a, HFA 227, or a mixture of HFA 227 and HFA 134a, all weights based on the total weight of the composition.

In one embodiment, included herein is a pharmaceutical composition in the form of an aerosol or an aerosolizable composition for inhalation administration, wherein the active agent consists of about 0.005% w/w to about 0.05% w/w of a glycopyrronium salt. The composition can include one or more pharmaceutically acceptable excipients.

In another embodiment, included herein is a pharmaceutical composition in the form of an aerosol or an aerosolizable composition for inhalation administration, wherein the active agent consists of about 1 μg to about 30 μg per actuation of glycopyrronium salt. The composition can include one or more pharmaceutically acceptable excipients.

In certain aspects, the compositions wherein the active agent consists of a glycopyrronium salt optionally include polyvinyl pyrrolidone, polyethylene glycol, a propellant or a combination thereof.

The propellant can be HFA 134a, HFA 227, or a mixture of HFA 227 and HFA 134a. In one aspect, the composition comprises at least about 30% w/w of the HFA 227 in the mixture of HFA 227 and HFA 134a, based upon the weight of the propellant. In one aspect, the composition comprises at least about 30% w/w of the HFA 134a in the mixture of HFA 227 and HFA 134a, based upon the weight of the propellant. In some embodiments, the ratio of HFA 227:HFA 134a is 0:100 to about 100:0, about 20:80 to about 80:20, about 40:60 to about 60:40, or about 30:70 to about 70:30. In other embodiments, the composition is substantially free of HFA 134a.

The polyethylene glycol, when present, is present in an amount of about 0.005% w/w to about 0.08% w/w, about 0.01% w/w to about 0.05% w/w, based upon the total weight of the composition. In a specific embodiment, the polyethylene glycol is present in an amount of 0.02% w/w and is polyethylene glycol 1000.

In one aspect, a pharmaceutical composition in the form of an aerosol or an aerosolizable composition for inhalation administration consists of about 0.005% w/w to about 0.05% w/w of a glycopyrronium salt, about 0.00001% w/w to about 0.01% w/w of polyvinyl pyrrolidone, about 0.005% w/w to about 0.08% w/w of polyethylene glycol, and a propellant selected from HFA 134a, HFA 227, and a mixture of HFA 227 and HFA 134a, all weights based on the total weight of the composition.

In any of the foregoing embodiments, the arformoterol salt may be arformoterol tartrate. In any of the foregoing embodiments, the glycopyrronium salt may be glycopyrronium bromide.

In certain embodiments, any of the pharmaceutical compositions described above may be free or substantially free of an organic acid or an inorganic acid. By free or substantially free, it is meant that no organic or inorganic acid is added to the compositions. For example, when a composition is substantially free of an excipient, such as substantially free of an organic acid, the composition typically comprises less than 0.1% w/w, or in certain embodiments less than 0.0001% w/w of the composition.

In certain embodiments, the compositions are free or substantially free of added lactose, trehalose, ethanol, distrearoylphosphatidylcholine (DPSC), phospholipids or a combination thereof.

In certain embodiments, the aerosol fixed dose pharmaceutical compositions described herein (e.g., containing an effective amount of arformoterol or its salt and/or an effective amount of a glycopyrronium salt) have at least 50%, at least 70% or at least 90% of the particles having a Mass Median Aerodynamic Diameter (MMAD) of no more than about 10 μm, or about 1 μm to about 5 μm, or about 0.05 μm to about 3 μm.

In certain embodiments, the aerosol fixed dose pharmaceutical compositions described herein (e.g., containing an effective amount of arformoterol or its salt and/or an effective amount of a glycopyrronium salt) have a fine particle fraction (FPF) of about 40% to about 90%, or about 50% to about 80%.

In certain embodiments, the aerosol fixed dose pharmaceutical compositions described herein (e.g., containing an effective amount of arformoterol or its salt and/or an effective amount of a glycopyrronium salt) have a fine particle dose (FPD) for arformoterol or its salt of about 0.001 μg to about 20 μg, about 0.01 μg to about 10 μg, or about 0.1 mcg to about 5 μg.

In certain embodiments, the aerosol fixed dose pharmaceutical compositions described herein (e.g., containing an effective amount of arformoterol or its salt and/or an effective amount of a glycopyrronium salt) have a fine particle dose (FPD) for glycopyrronium salt of about 0.01 mcg to about 20 about 0.1 μg to about 15 or about 1 μg to about 10 μg.

The fixed dose pharmaceutical aerosol compositions described above are found to be stable by visual observation when stored at ambient (e.g., about 25° C. and a relative humidity (RH) of about 60%) or at accelerated conditions (e.g., at about 40° C. and about 75% RH) for at least about 30 minutes, 2 hours, 6 hours, 12 hours, 24 hours, or longer.

These compositions also exhibited good dose content uniformity (DCU) when analyzed for the top, middle and end dose.

In certain embodiments, the compositions described herein are in the form of a suspension. Suspensions preferably have only one phase (i.e., they are preferably a single phase suspension). In another embodiment, there is provided a fixed dose pharmaceutical composition in the form of an aerosol or an aerosolizable composition for inhalation administration comprising about 0.0005% w/w to about 0.05% w/w of arformoterol tartrate, about 0.005% w/w to about 0.05% w/w of glycopyrronium bromide, about 0.02% w/w of a polyethylene glycol (e.g., PEG 1000), and about 0.00001% w/w to about 0.01% w/w, or about 0.0001% w/w of polyvinyl pyrrolidone, all based upon the total weight of the composition.

In another embodiment, a pharmaceutical composition in the form of an aerosol or an aerosolizable composition for inhalation administration consists of (a) about 0.0001% w/w to about 0.1% w/w, from about 0.0005% w/w to about 0.05% w/w of arformoterol or its salt, (b) about 0.005 w/w to about 0.08% w/w, or about 0.01% w/w to about 0.05 w/w of a polyethylene glycol having a molecular weight of 200 to 6000 (e.g., PEG 1000), and (c) about 0.00001% w/w to about 0.01% w/w, or about 0.00005% w/w to about 0.001% w/w of povidone, all based upon the total weight of the composition.

In an aspect, a stable pharmaceutical composition in the form of an aerosol or an aerosolizable composition for inhalation administration consists of (a) about 0.0005% w/w to about 0.05% w/w of arformoterol tartrate, (b) about 0.005% w/w to about 0.05% w/w, or from about 0.01% w/w to 0.03% w/w of polyethylene glycol 1000, and (c) about 0.00001% w/w to about 0.01% w/w, or from about 0.00005% w/w to 0.001% w/w of povidone based upon total weight of the composition.

In a more specific embodiment, a pharmaceutical composition in the form of an aerosol or an aerosolizable composition for inhalation administration consists of (a) about 0.0005% w/w to about 0.05% w/w of arformoterol or its salt, (b) about 0.02% w/w of polyethylene glycol 1000, and (c) about 0.00005% w/w to 0.001% w/w or about 0.0001% w/w of povidone, all based upon the total weight of the composition.

In another embodiment, there is provided a pharmaceutical composition in the form of an aerosol or an aerosolizable composition for inhalation administration consisting of at least about 0.001% w/w of arformoterol tartrate, about 0.02% w/w of a polyethylene glycol (e.g., PEG 1000) and about 0.00005 w/w to 0.001% w/w, or about 0.0001% w/w of povidone, based upon the total weight of the composition.

In another embodiment, a pharmaceutical composition in the form of an aerosol or an aerosolizable composition for inhalation administration consists of (a) about 0.0001% w/w to about 0.1% w/w, about 0.005% w/w to about 0.05% w/w of a glycopyrronium salt, (b) about 0.005% w/w to about 0.08% w/w, about 0.01% w/w to about 0.05% w/w, of a polyethylene glycol having a molecular weight from 200 to 6000 (e.g., PEG 1000), and (c) about 0.00001% w/w to about 0.01% w/w, about 0.00005% w/w to 0.001% w/w, of povidone, all based upon the total weight of the composition.

In an aspect, a stable pharmaceutical composition in the form of an aerosol or an aerosolizable composition for inhalation administration consists of (a)) about 0.005% w/w to about 0.05% w/w, of glycopyrronium bromide, (b) 0.005% w/w to about 0.05% w/w, or from about 0.01% w/w to about 0.03% w/w, of polyethylene glycol 1000, and (c) 0.00001% w/w to about 0.01% w/w, or from about 0.00005% w/w to about 0.001% w/w, of povidone based upon total weight of the composition.

In a more specific embodiment, a pharmaceutical composition in the form of an aerosol or an aerosolizable composition for inhalation administration consists of (a) about 0.005% w/w to about 0.05% w/w of a glycopyrronium salt, (b) about 0.02% w/w of polyethylene glycol 1000, and (c) about 0.00005% w/w to about 0.001% w/w, or about 0.0001% w/w of povidone, all based upon the total weight of the composition.

In another embodiment, there is provided a pharmaceutical composition in the form of an aerosol or an aerosolizable composition for inhalation administration consisting of at least about 0.005% w/w of glycopyrronium bromide, about 0.02% w/w of a polyethylene glycol (e.g., PEG 1000) and about 0.00005 w/w to about 0.001% w/w, or about 0.0001% w/w of povidone, all based upon the total weight of the composition.

In another embodiment, there is provided a stable fixed dose suspension in the form of an aerosol or an aerosolizable composition for inhalation administration comprising (a) about 0.0005% w/w to about 0.05, % w/w of arformoterol or its salt, e.g. arformoterol tartrate, (b) about 0.005% w/w to about 0.05% w/w, of a glycopyrronium salt, e.g., glycopyrronium bromide, (c) about 0.02% w/w, of polyethylene glycol 1000, (d) less than about 0.0015% w/w, or about 0.00005% w/w to about 0.001% w/w, of povidone (e.g., PVP K25, PVP K30, PVP K60, PVP K90) and (e) a propellant selected from HFA 134a, HFA 227 or a mixture of HFA 227 and HFA 134a.

In another embodiment, there is provided a stable fixed dose suspension in the form of an aerosol or an aerosolizable composition for inhalation administration comprising (a) about 0.0005% w/w to about 0.05% w/w, of arformoterol or its salt, e.g. arformoterol tartrate, (b) about 0.005% w/w to about 0.05% w/w, of a glycopyrronium salt, e.g., glycopyrronium bromide, (c) about 0.02% w/w, of polyethylene glycol 1000, (d) about 0.0001% w/w of povidone (e.g., PVP K25) and (e) a propellant selected from HFA 134a, HFA 227 or a mixture of HFA 227 and HFA 134a.

In another embodiment, there is provided a stable suspension in the form of an aerosol or an aerosolizable composition for inhalation administration consisting of (a) about 0.0005% w/w to about 0.05% w/w, of arformoterol or its salt, e.g. arformoterol tartrate, (b) about 0.02% w/w, of polyethylene glycol 1000, (c) less than about 0.001% w/w, or about 0.00005 w/w to 0.001% w/w, of povidone (e.g., PVP K25, PVP K30, PVP K60, PVP K90) and (d) a propellant selected from HFA 134a, HFA 227 or a mixture of HFA 227 and HFA 134a.

In another embodiment, there is provided a stable suspension in the form of an aerosol or an aerosolizable composition for inhalation administration consisting of (a) about 0.005% w/w to about 0.05% w/w, of a glycopyrronium salt, e.g., glycopyrronium bromide, (b) about 0.02% w/w, of polyethylene glycol 1000, (c) less than about 0.001% w/w, or about 0.00005 w/w to 0.001% w/w, of povidone and (d) a propellant selected from HFA 134a, HFA 227 or a mixture of HFA 227 and HFA 134a.

In another embodiment, there is provided a stable fixed dose composition in the form of an aerosol or an aerosolizable composition for inhalation administration comprising (a) about 0.0005% w/w to about 0.05, % w/w of arformoterol or its salt, e.g. arformoterol tartrate, (b) about 0.005% w/w to about 0.05% w/w, of a glycopyrronium salt, e.g., glycopyrronium bromide, and (c) a propellant selected from HFA 134a, HFA 227 or a mixture of HFA 227 and HFA 134a.

In another embodiment, there is provided a stable fixed dose composition in the form of an aerosol or an aerosolizable composition for inhalation administration comprising (a) about 0.0005% w/w to about 0.05, % w/w of arformoterol or its salt, e.g. arformoterol tartrate, (b) about 0.005% w/w to about 0.05% w/w, of a glycopyrronium salt, e.g., glycopyrronium bromide, (c) about 0.02% w/w, of polyethylene glycol 1000, and (d) a propellant selected from HFA 134a, HFA 227 or a mixture of HFA 227 and HFA 134a.

In another embodiment, there is provided a stable fixed dose composition in the form of an aerosol or an aerosolizable composition for inhalation administration comprising (a) about 0.0005% w/w to about 0.05, % w/w of arformoterol or its salt, e.g. arformoterol tartrate, (b) about 0.005% w/w to about 0.05% w/w, of a glycopyrronium salt, e.g., glycopyrronium bromide, (c) about 0.0001% w/w, of povidone (e.g., PVP K25, PVP K30, PVP K60, PVP K90) and (d) a propellant selected from HFA 134a, HFA 227 or a mixture of HFA 227 and HFA 134a.

In another embodiment, there is provided a stable fixed dose composition in the form of an aerosol or an aerosolizable composition for inhalation administration comprising (a) about 0.0040% w/w of arformoterol or its salt, (b) about 0.0138% w/w of glycopyrronium salt, (c) about 0.02% w/w of polyethylene glycol 1000, (d) about 0.0001% w/w of polyvinyl pyrrolidone (e.g., PVP K25), and (e) a propellant selected from HFA 134a, HFA 227, and a mixture of HFA 227 and HFA 134a, wherein all weights are based upon the total weight of the pharmaceutical composition.

In another embodiment, there is provided a stable fixed dose composition in the form of an aerosol or an aerosolizable composition for inhalation administration comprising (a) about 0.0041% w/w of arformoterol or its salt, (b) about 0.0142% w/w of glycopyrronium salt, (c) about 0.02% w/w of polyethylene glycol 1000, (d) about 0.0001% w/w of polyvinyl pyrrolidone (e.g., PVP K25), and (e) a propellant selected from HFA 134a, HFA 227, and a mixture of HFA 227 and HFA 134a, wherein all weights are based upon the total weight of the pharmaceutical composition.

In another embodiment, there is provided a stable fixed dose composition in the form of an aerosol or an aerosolizable composition for inhalation administration comprising (a) about 0.0042% w/w of arformoterol or its salt, (b) about 0.0146% w/w of glycopyrronium salt, (c) about 0.02% w/w of polyethylene glycol 1000, (d) about 0.0001% w/w of polyvinyl pyrrolidone (e.g., PVP K25), and (e) a propellant selected from HFA 134a, HFA 227, and a mixture of HFA 227 and HFA 134a, wherein all weights are based upon the total weight of the pharmaceutical composition.

In another embodiment, there is provided a stable fixed dose composition in the form of an aerosol or an aerosolizable composition for inhalation administration comprising (a) about 0.0043% w/w of arformoterol or its salt, (b) about 0.0149% w/w of glycopyrronium salt, (c) about 0.02% w/w of polyethylene glycol 1000, (d) about 0.0001% w/w of polyvinyl pyrrolidone (e.g., PVP K25), and (e) a propellant selected from HFA 134a, HFA 227, and a mixture of HFA 227 and HFA 134a, wherein all weights are based upon the total weight of the pharmaceutical composition.

In another embodiment, there is provided a stable fixed dose composition in the form of an aerosol or an aerosolizable composition for inhalation administration comprising (a) about 0.0038% w/w of arformoterol or its salt, (b) about 0.0134% w/w of glycopyrronium salt, and (c) a propellant selected from HFA 134a, HFA 227, and a mixture of HFA 227 and HFA 134a, wherein all weights are based upon the total weight of the pharmaceutical composition.

In another embodiment, there is provided a stable fixed dose composition in the form of an aerosol or an aerosolizable composition for inhalation administration comprising (a) about 0.0038% w/w of arformoterol or its salt, (b) about 0.0134% w/w of glycopyrronium salt, (c) about 0.02% w/w, of polyethylene glycol 1000, and (d) a propellant selected from HFA 134a, HFA 227 or a mixture of HFA 227 and HFA 134a, wherein all weights are based upon the total weight of the pharmaceutical composition.

In another embodiment, there is provided a stable fixed dose composition in the form of an aerosol or an aerosolizable composition for inhalation administration comprising (comprising (a) about 0.0038% w/w of arformoterol or its salt, (b) about 0.0134% w/w of glycopyrronium salt, (c) about 0.0001% w/w, of povidone (e.g., PVP K25, PVP K30, PVP K60, PVP K90) and (d) a propellant selected from HFA 134a, HFA 227 or a mixture of HFA 227 and HFA 134a, wherein all weights are based upon the total weight of the pharmaceutical composition.

In another embodiment, there is provided a fixed dose pharmaceutical composition in the form of an aerosol or an aerosolizable composition for inhalation administration comprising (a) arformoterol tartrate in an amount of 0.01 μg to 50 μg, (b) glycopyrronium bromide in an amount of 0.1 μg to 50 μg, (c) polyethylene glycol 1000, (d) povidone, and (e) a propellant selected from HFA 134a, HFA 227 or a mixture of HFA 227 and HFA 134a.

In another embodiment, there is provided a fixed dose pharmaceutical composition in the form of an aerosol or an aerosolizable composition for inhalation administration comprising (a) arformoterol tartrate in an amount selected from about 0.5 μg, about 0.75 μg, about 1 μg, about 1.5 μg, about 3 μg and about 6 μg (b) glycopyrronium bromide in an amount selected from about 1.5 μg, about 3.75 μg, about 7.5 μg, about 11.25 μg and about 15 μg (c) polyethylene glycol 1000, (d) povidone and (e) a propellant selected from HFA 134a, HFA 227 or a mixture of HFA 227 and HFA 134a.

In another embodiment, there is provided a pharmaceutical composition in the form of an aerosol or an aerosolizable composition for inhalation administration consisting of (a) at least about 0.0009% w/w of arformoterol tartrate, (b) about 0.02% w/w of polyethylene glycol (e.g., PEG 1000), (c) about 0.0001% w/w of povidone, and (d) a propellant selected from HFA 134a, HFA 227 or a mixture of HFA 227 and HFA 134a.

In another embodiment, there is provided a pharmaceutical composition in the form of an aerosol or an aerosolizable composition for inhalation administration consisting of (a) about 0.0013% w/w of arformoterol tartrate, (b) about 0.02% w/w of polyethylene glycol (e.g., PEG 1000), (c) about 0.0001% w/w of povidone, and (d) a propellant selected from HFA 134a, HFA 227 or a mixture of HFA 227 and HFA 134a.

In another embodiment, there is provided a stable pharmaceutical composition in the form of an aerosol or an aerosolizable composition for inhalation administration consisting of (a) about 0.0025 w/w of arformoterol tartrate, (b) about 0.02% w/w of polyethylene glycol 1000, (c) about 0.0001% w/w of povidone, and (d) HFA 227.

In another embodiment, there is provided a stable pharmaceutical composition in the form of an aerosol or an aerosolizable composition for inhalation administration consisting of (a) about 0.0038% w/w of arformoterol tartrate, (b) about 0.02% w/w of polyethylene glycol 1000, (c) about 0.0001% w/w of povidone, and (d) HFA 227.

In another embodiment, there is provided a stable pharmaceutical composition in the form of an aerosol or an aerosolizable composition for inhalation administration consisting of (a) about 0.0075 w/w of arformoterol tartrate, (b) about 0.02% w/w of polyethylene glycol 1000, (c) about 0.0001% w/w of povidone, and (d) HFA 227.

In another embodiment, there is provided a stable pharmaceutical composition in the form of an aerosol or an aerosolizable composition for inhalation administration consisting of (a) about 0.0026% w/w of arformoterol tartrate, (b) about 0.02% w/w of polyethylene glycol 1000, (c) about 0.0001% w/w of povidone, and (d) a propellant selected from HFA 134a, HFA 227 or a mixture of HFA 227 and HFA 134a.

In another embodiment, there is provided a stable pharmaceutical composition in the form of an aerosol or an aerosolizable composition for inhalation administration consisting of (a) about 0.0038% w/w of arformoterol tartrate, (b) about 0.02% w/w of polyethylene glycol 1000, (c) about 0.0001% w/w of povidone, and (d) a propellant selected from HFA 134a, HFA 227 or a mixture of HFA 227 and HFA 134a.

In another embodiment, there is provided a stable pharmaceutical composition in the form of an aerosol or an aerosolizable composition for inhalation administration consisting of (a) about 0.0077% w/w of arformoterol tartrate, (b) about 0.02% w/w of polyethylene glycol 1000, (c) about 0.0001% w/w of povidone, and (d) a propellant selected from HFA 134a, HFA 227 or a mixture of HFA 227 and HFA 134a.

In another embodiment, there is provided a pharmaceutical composition in the form of an aerosol or an aerosolizable composition for inhalation administration consisting of (a) at least about 0.005% w/w of glycopyrronium bromide, (b) about 0.02% w/w of polyethylene glycol (e.g., PEG 1000), (c) about 0.0001% w/w of povidone, and (d) a propellant selected from HFA 134a, HFA 227 or a mixture of HFA 227 and HFA 134a.

In another embodiment, there is provided a stable pharmaceutical composition in the form of an aerosol or an aerosolizable composition for inhalation administration consisting of (a) about 0.0065% w/w of glycopyrronium bromide, (b) about 0.02% w/w of polyethylene glycol 1000, (c) about 0.0001% w/w of povidone, and (d) HFA 227.

In another embodiment, there is provided a stable pharmaceutical composition in the form of an aerosol or an aerosolizable composition for inhalation administration consisting of (a) about 0.0131% w/w of glycopyrronium bromide, (b) about 0.02% w/w of polyethylene glycol 1000, (c) about 0.0001% w/w of povidone, and (d) HFA 227.

In another embodiment, there is provided a stable pharmaceutical composition in the form of an aerosol or an aerosolizable composition for inhalation administration consisting of (a) about 0.0196% w/w of glycopyrronium bromide, (b) about 0.02% w/w of polyethylene glycol 1000, (c) about 0.0001% w/w of povidone, and (d) HFA 227.

In another embodiment, there is provided a stable pharmaceutical composition in the form of an aerosol or an aerosolizable composition for inhalation administration consisting of (a) about 0.0067% w/w of glycopyrronium bromide, (b) about 0.02% w/w of polyethylene glycol 1000, (c) about 0.0001% w/w of povidone, and (d) a propellant selected from HFA 134a, HFA 227 or a mixture of HFA 227 and HFA 134a.

In another embodiment, there is provided a stable pharmaceutical composition in the form of an aerosol or an aerosolizable composition for inhalation administration consisting of (a) about 0.0134% w/w of glycopyrronium bromide, (b) about 0.02% w/w of polyethylene glycol 1000, (c) about 0.0001% w/w of povidone, and (d) a propellant selected from HFA 134a, HFA 227 or a mixture of HFA 227 and HFA 134a.

In another embodiment, there is provided a stable pharmaceutical composition in the form of an aerosol or an aerosolizable composition for inhalation administration consisting of (a) about 0.0201% w/w of glycopyrronium bromide, (b) about 0.02% w/w of polyethylene glycol 1000, (c) about 0.0001% w/w of povidone, and (d) a propellant selected from HFA 134a, HFA 227 or a mixture of HFA 227 and HFA 134a.

In any of the foregoing embodiments, the amount of arformoterol tartrate in the compositions can be 0.01 μg to 50 μg and the amount of glycopyrronium bromide can be 0.1 μg to 50 μg.

In another embodiment, there is provided a pharmaceutical composition in the form of an aerosol or an aerosolizable composition for inhalation administration consisting of (a) arformoterol tartrate in an amount of 0.01 μg to 50 μg, or in an amount of 0.1 μg to 7.5 μg, (b) polyethylene glycol 1000, (c) povidone, and (d) a propellant selected from HFA 134a, HFA 227 or a mixture of HFA 227 and HFA 134a.

In another embodiment, there is provided a pharmaceutical composition in the form of an aerosol or an aerosolizable composition for inhalation administration consisting of (a) glycopyrronium bromide in an amount of 0.1 μg to 50 μg, or in an amount of 1 μg to 30 μg, (b) polyethylene glycol 1000, (c) povidone, and (d) a propellant selected from HFA 134a, HFA 227 or a mixture of HFA 227 and HFA 134a.

Exemplary amounts of arformoterol tartrate for the compositions described herein are about 0.5 μg, about 1 μg, about 1.5 μg, about 3 μg and about 6 μg. Exemplary amounts of glycopyrronium bromide for the compositions described herein are about 1.5 μg, about 3.75 μg, about 7.5 μg, about 11.25 μg and about 15 μg.

In another embodiment, there is provided a pharmaceutical composition in the form of an aerosol or an aerosolizable composition for inhalation administration consisting of (a) arformoterol or its salt selected from about 0.5 μg, about 1 μg, about 1.5 μg, about 3 μg and about 6 μg (b) polyethylene glycol 1000, (c) povidone and (d) a propellant selected from HFA 134a, HFA 227 or a mixture of HFA 227 and HFA 134a.

In another embodiment, there is provided a pharmaceutical composition in the form of an aerosol or an aerosolizable composition for inhalation administration consisting of (a) a glycopyrronium salt selected from about 1.5 μg, about 3.75 μg, about 7.5 μg, about 11.25 μg and about 15 μg (b) polyethylene glycol 1000, (c) povidone and (d) a propellant selected from HFA 134a, HFA 227 or a mixture of HFA 227 and HFA 134a.

In another embodiment, a stable suspension in the form of an aerosol or an aerosolizable composition for inhalation administration to a human comprises (a) arformoterol or its salt in an amount of about 0.1 μg to about 7.5 μg, (b) a glycopyrronium salt in the amount of about 1 μg to about 30 μg, (c) about 0.0001% w/w of excipient present in substantially insoluble form, (d) about 0.02% w/w of excipient present in soluble form, and (e) at least one propellant.

In an aspect, the total weight of arformoterol or its salt and/or glycopyrrolate salt is equal to or more than excipient present in substantially insoluble form; wherein the weight of arformoterol or its salt and/or the glycopyrrolate salt is equal to or less than the excipient present in soluble form; wherein the composition is free or substantially free of an organic acid or an inorganic acid.

In another embodiment, the present invention relates to a stable suspension in the form of an aerosol or an aerosolizable composition for inhalation administration to a human consisting of (a) a glycopyrronium salt in the amount of about 1 μg to about 30 μg, (b) about 0.0001% w/w of excipient present in substantially insoluble form, (c) about 0.02% w/w of excipient present in soluble form, and (d) at least one propellant.

In an aspect, the weight of a glycopyrronium salt is equal to or more than excipient present in substantially insoluble form; wherein the weight of a glycopyrronium salt is equal to or less than the excipient present in soluble form; wherein the composition is free or substantially free of an organic acid or an inorganic acid.

In another embodiment, the present invention relates to a stable suspension in the form of an aerosol or an aerosolizable composition for inhalation administration to a human consisting of (a) arformoterol or its salt in an amount of about 0.1 μg to about 7.5 μg, (b) about 0.0001% w/w of excipient present in substantially insoluble form, (c) about 0.02% w/w of excipient present in soluble form, and (d) at least one propellant.

In an aspect, the weight of arformoterol or its salt is equal to or more than excipient present in substantially insoluble form; wherein the weight of arformoterol or its salt is equal to or less than the excipient present in soluble form; wherein the composition is free or substantially free of an organic acid or an inorganic acid.

In another embodiment, a stable suspension in the form of aerosol or an aerosolizable composition for inhalation administration to a human comprises (a) an active ingredient selected from arformoterol or its salt in an amount of about 0.1 μg to about 7.5 μg, a glycopyrronium salt in the amount of about 1 μg to about 30 μg or a combination thereof, (b) about 0.0001% w/w of povidone (PVP), (c) about 0.02% w/w of polyethylene glycol 1000, and (d) a propellant selected from HFA 134a, HFA 227 or a mixture of HFA 227 and HFA 134a wherein the ratio of arformoterol or its salt and a glycopyrronium salt is in the range of about 1:0.70 to about 1:30. In one aspect, the total weight of arformoterol or its salt and a glycopyrronium salt is equal to or more than the weight of povidone (PVP). In another aspect, the weight of arformoterol or its salt or a glycopyrronium salt is equal to or less than the weight of polyethylene glycol 1000.

In another embodiment, a stable suspension in the form of an aerosol or an aerosolizable composition for inhalation administration to a human consists of (a) arformoterol or its salt in an amount of about 0.1 μg to about 7.5 μg, (b) about 0.0001% w/w of povidone (PVP), (c) about 0.02% w/w of polyethylene glycol 1000, and (d) a propellant selected from HFA 134a, HFA 227 or a mixture of HFA 227 and HFA 134a. In one aspect, the weight of arformoterol or its salt is equal to or more than the weight of povidone (PVP). In another aspect, the weight of arformoterol or its salt is equal to or less than the weight of polyethylene glycol 1000. In another aspect, the composition is free or substantially free of an organic acid or an inorganic acid.

In another embodiment, the present invention relates to a stable suspension in the form of an aerosol or an aerosolizable composition for inhalation administration to a human consists of (a) a glycopyrronium salt in the amount of about 1 μg to about 30 μg, (b) about 0.0001% w/w of povidone (PVP) (c) about 0.02% w/w of polyethylene glycol 1000 and (d) a propellant selected from HFA 134a, HFA 227 or a mixture of HFA 227 and HFA 134a.

In one aspect, the weight of a glycopyrronium salt is equal to or more than the weight of povidone (PVP). In another aspect, the weight of the glycopyrronium salt is equal to or less than the weight of polyethylene glycol 1000. In another aspect, the composition is free or substantially free of an organic acid or an inorganic acid.

The pharmaceutical compositions described herein can include a propellant selected from HFA 134a (also known as hydrofluoroalkane (HFA) 134a or 1,1,1,2-tetrafluoroethane), HFC-227 (HFA 227 or HFA-227ea or 1,1,1,2,3,3,3-heptafluoropropane), or a combination thereof. In an aspect, the pharmaceutical compositions include at least about 30% w/w, greater than 40% w/w, about 35% w/w to about 60% w/w, or from about 40% w/w to about 50% w/w of HFA 227, based upon the total weight of the propellant. In an aspect, the pharmaceutical compositions include at least about 30% w/w, greater than 40% w/w, about 35% w/w to about 60% w/w, or from about 40% w/w to about 50% w/w of HFA 134a, based upon the total weight of the propellant.

The compositions may comprise one or more pharmaceutically acceptable excipients such as, but not limited to, polymers, propellants, lubricants, surfactants, stabilizing agents, suspending agents, dispersing agents, co-solvents, chelating agents, osmotic agents, bulking agents, non-volatile components, buffers/pH adjusting agents, surface active agents, preservatives, complexing agents, or combinations thereof.

In addition to HFA-134a and HFA-227, propellants include difluoromethane (HFC-32), 1,1,1-trifluoroethane HFC-I43 (a)), 1,1,2,2-tetrafluoroethane HFC-134), and 1, 1-difluoroethane HFC-152(a)), and such other propellants which may be known in the art, which may be used alone or in combination.

Lubricants can be included in the composition in an amount desired and which, when added, provides lubrication and prevents the drugs from sticking to the inside surface of the manufacturing vessel. In one embodiment, the lubricant comprises one or more polyethylene glycols (PEG) such as, but not limited to, PEG 300, PEG 400, PEG 1000, and combinations thereof. In another embodiment, polyethylene glycol 1000 (PEG 1000) is used as a lubricant.

Dispersing agents can be included in the composition in an amount desired and which, when added, provides easy redispersion of settled particles on standing. Surfactants can act as dispersing agents. In one embodiment, the dispersing agent comprises one or more of polyvinyl pyrrolidone such as, but not limited to, povidone K25 or povidone K30.

A co-solvent is a solvent which is miscible in the composition in the amount desired and which, when added, provides a composition in which the drug(s) can be dissolved. The function of the co-solvent is to increase the solubility of the drug(s) and the excipients in the composition.

In one embodiment, the co-solvent comprises one or more of C2-C6 aliphatic alcohols (such as, but not limited to, ethyl alcohol and isopropyl alcohol), glycerol, polyoxyethylene alcohols, polyoxyethylene fatty acid esters, hydrocarbons (such as, but not limited to, n-propane, n-butane, isobutane, n-pentane, iso-pentane, neo-pentane, and n-hexane), ethers (such as but not limited to diethyl ether), and combinations thereof.

Exemplary surfactants may be employed in the aerosol composition, including those intended for administration through metered dose inhalers, which may serve to stabilize the aerosol composition and improve the performance. The surfactant may comprise one or more ionic and/or non-ionic surfactants including, but not limited to, salts of stearic acids such as magnesium stearate, esters such as ascorbyl palmitate, isopropyl myristate and tocopherol esters, oleic acid, lecithin, tyloxapol, polysorbates such as polysorbate 80, polysorbate 20, and polysorbate 40, vitamin E-TPGS, macrogol hydroxystearates such as macrogol-15-hydroxystearate, acetylated monoglycerides such as Myvacet 9-45 and Myvacet 9-08, polyoxyethylene ethers, ethyloleate, glyceryl trioleate, glyceryl monolaurate, glyceryl monooleate, glyceryl monosterate, glyceryl monoricinoleate, cetyl alcohol, steryl alcohol, cetylpyridinium chloride, block polymers, natural oils, polyvinyl pyrrolidone, sorbitan fatty acid esters such as sorbitan trioleate, polyethoxylated sorbitan fatty acid esters (for example polyethoxylated sorbitan trioleate), sorbimacrogol oleate, synthetic amphotensides (tritons), ethylene oxide ethers of octylphenolformaldehyde condensation products, phosphatides such as lecithin, polyethoxylated fats, polyethoxylated oleotriglycerides and polyethoxylated fatty alcohols, and combinations thereof.

The surfactants may also be selected from others known in the art including, but not limited to, oils such as corn oil, olive oil, cottonseed oil and sunflower seed oil, mineral oils such as liquid paraffin, oleic acid, phospholipids such as lecithin, sorbitan fatty acid esters such as sorbitan trioleate, Tween 20, Tween 60, Tween 80, PEG-25 glyceryl trioleate, PVP, citric acid, and PFDA (per fluoro-n-decanoic acid), and combinations thereof.

The pharmaceutical composition may include a non-volatile component. The non-volatile component is the suspended or dissolved constituents that would be left after evaporation of the solvent. The non-volatile component may comprise one or more monosaccharides such as glucose; arabinose; disaccharides such as lactose and maltose; oligosaccharides and polysaccharides such as dextrans; polyalcohol such glycerol, sorbitol, mannitol, and xylitol; and salts such as potassium chloride, magnesium chloride, magnesium sulphate, sodium chloride, sodium citrate, sodium phosphate, sodium hydrogen phosphate, sodium hydrogen carbonate, potassium citrate, potassium phosphate, potassium hydrogen phosphate, potassium hydrogen carbonate, calcium carbonate, calcium chloride, and combinations thereof.

Bulking agents may be employed for example, for a composition intended for metered dose inhalation. The bulking agent may comprise one or more saccharides, including monosaccharides, disaccharides, polysaccharides and sugar alcohols such as arabinose, glucose, fructose, ribose, mannose, sucrose, trehalose, lactose, maltose, starches, dextran, mannitol, and combinations thereof.

Buffers or pH adjusting agents may be employed, for example, for a composition intended for metered dose inhalation. The buffer or the pH adjusting agent may comprise one or more of organic or inorganic acids such as citric acid, ascorbic acid, hydrochloric acid, sulfuric acid, nitric acid, phosphoric acid, and combinations thereof.

Preservatives may be employed in the compositions to protect the composition from contamination with pathogenic bacteria. The preservative may comprise one or more of benzalkonium chloride, benzoic acid, benzoates such as sodium benzoate and such other preservatives which may be known in the art, and combinations thereof.

Exemplary complexing agents may be employed in the aerosol compositions which are capable of forming complex bonds. The complexing agent may comprise one or more of sodium EDTA or disodium EDTA.

In one embodiment, the polymer may be a homopolymer, that is the polymer comprises of the same recurring structural units, or it may be a copolymer, that is the polymer contains recurring units in addition to either amide containing units or carboxylic acid ester units. The polymer may also be a copolymer of amide containing units and carboxylic acid ester units. Such copolymers may be either block copolymers or random copolymers; preferably the polymer is recurring structural units containing an amide group such as polyvinylpyrrolidone or carboxylic acid ester such as polyvinylacetate. In an aspect of the invention, addition of polymer results in stabilization of the composition. In some embodiments polymer is present is about 0.00001% w/w, to about 0.01% w/w, or about 0.00005% w/w to about 0.001% w/w, based upon the total weight of the composition.

The aerosol or an aerosolizable composition for inhalation may be administered in the form of a metered dose inhaler, for example.

In one embodiment, a method of treating a respiratory disorder (e.g., asthma or COPD) in a subject comprises administering by inhalation to the subject a fixed dose pharmaceutical composition in the form of an aerosol for inhalation administration comprising an effective amount of arformoterol or its salt and an effective amount of glycopyrronium bromide, in any of the embodiments described herein. The compositions may contain polyvinyl pyrrolidone, polyethylene glycol, a propellant, or a combination thereof.

In an embodiment, a method of treating a respiratory disorder (e.g., asthma or COPD) in a subject comprises administering by inhalation to the subject a pharmaceutical composition in the form of an aerosol for inhalation administration wherein the active agent consists of an effective amount arformoterol or its salt, in any of the embodiments described herein. The compositions may contain polyvinyl pyrrolidone, polyethylene glycol, a propellant, or a combination thereof.

In an embodiment, a method of treating a respiratory disorder (e.g., asthma or COPD) in a subject comprises administering by inhalation to the subject a pharmaceutical composition in the form of an aerosol for inhalation administration wherein the active agent consists of an effective amount of glycopyrronium salt, in any of the embodiments described herein. The compositions may contain polyvinyl pyrrolidone, polyethylene glycol, a propellant, or a combination thereof.

Respiratory disorders, include but are not limited to asthma, emphysema, bronchitis, COPD, sinusitis, respiratory depression, reactive airways dysfunction syndrome (RADS), acute respiratory distress syndrome (ARDS), irritant induced asthma, occupational asthma, sensory hyper-reactivity, airway (or pulmonary) inflammation, multiple chemical sensitivity, or aid in smoking cessation therapy.

In a further embodiment, described herein is the use of an effective amount of arformoterol or its salt and a glycopyrronium salt in the preparation of a fixed dose pharmaceutical composition in the form of an aerosol for inhalation administration for the treatment of a respiratory disorder in a subject. The composition is any of the embodiments described herein. The compositions may contain polyvinyl pyrrolidone, polyethylene glycol, a propellant, or a combination thereof.

In a further embodiment, described herein is the use of an effective amount of arformoterol or its salt in the form of an aerosol for inhalation administration for the treatment of a respiratory disorder in a subject. The composition is any of the embodiments described herein. The compositions may contain polyvinyl pyrrolidone, polyethylene glycol, a propellant, or a combination thereof.

In a further embodiment, described herein is the use of an effective amount of a glycopyrronium salt in the form of an aerosol for inhalation administration for the treatment of a respiratory disorder in a subject. The composition is any of the embodiments described herein. The compositions may contain polyvinyl pyrrolidone, polyethylene glycol, a propellant, or a combination thereof.

In a further embodiment, the present invention relates to a fixed dose pharmaceutical composition in the form of an aerosol for inhalation administration comprising (a) a therapeutically effective amount of arformoterol or its salt, (b) a therapeutically effective amount of glycopyrronium bromide, (c) polyethylene glycol, (d) povidone, and (e) a propellant for the treatment of respiratory disorders in a subject.

In a further embodiment, the present invention relates to a pharmaceutical composition in the form of an aerosol for inhalation administration consisting of (a) an effective amount of arformoterol or its salt, (b) polyethylene glycol, (c) povidone, and (d) a propellant for the treatment of respiratory disorders in a subject.

In a further embodiment, the present invention relates to a pharmaceutical composition in the form of an aerosol for inhalation administration consisting of (a) an effective amount of glycopyrronium bromide, (b) polyethylene glycol, (c) povidone, and (d) a propellant for the treatment of respiratory disorders in a subject.

There are a number of routinely applied analytical tests for aerosol dosage forms for inhalation administration, including mass median aerodynamic diameter (MMAD), fine particle dose or fraction (FPD or FPF), and geometric standard deviation (GSD).

The Mass Median Aerodynamic Diameter (MMAD) is defined as the diameter at which 50% of the particles by mass are larger and 50% are smaller.

The fine particle fraction (FPF) is the fraction of emitted particles that are less than 5 μm in aerodynamic diameter.

The Geometric Standard Deviation (GSD) is a measure of the spread of an aerodynamic particle size distribution. Typically calculated as follows:

GSD=(d₈₄/d₁₆)^{1/2}

where d₈₄ and d₁₆ represent the diameters at which 84% and 16% of the aerosol mass are contained, respectively, in diameters less than these diameters.

The fine particle dose (FPD) is defined as the mass of active pharmaceutical ingredient per actuation of the inhaler contained in particles finer than 5.0 μm aerodynamic diameter

Out of these, the MMAD is probably the most widespread, although acceptance criteria are typically based on fine particle dose (FPD) applicable to the active. The content uniformity in inhalers may be evaluated by tests such as uniformity of delivered dose (UODD), in which the composition is assayed at initial, middle and end points of the aerosol from a metered dose inhaler.

Various analytical tests including, but not limited to, MMAD, FPD, FPF and GSD can be measured by various instruments such as Anderson Cascade Impactor, a device that uses a series of impaction stages with decreasing particle cut size so that particles can be separated into relatively narrow intervals of aerodynamic diameter.

The aerosolizable composition may be packed in a suitable container for administration.

In another embodiment, there is provided a manufacturing process for preparing an inhalation suspension. The process comprises dispersing the drugs in polyethylene glycol, polyvinyl pyrrolidone and/or a co-solvent, and optionally adding chelating agents, osmotic agents and any other suitable ingredients. The propellant is added to the above mentioned blend and filled into a suitable container.

In one embodiment, disclosed is a method of preparing a fixed dose pharmaceutical composition comprising (a) arformoterol or its salt and/or a glycopyrronium salt thereof, and pharmaceutically acceptable excipients; wherein the arformoterol or its salt and/or the glycopyrronium salt is dispensed under an isolator having RH below 50%, below 30%, below 15%, more preferably below 5%, and a temperature of 10-40° C., 15-35° C., or 20-25° C.

The terms used herein are defined as follows. If a definition set forth in the present application and a definition set forth in a provisional application from which priority is claimed are in conflict, the definition in the present application shall control the meaning of the terms.

“Fixed dose” means that the composition is a combination composition in which both active agents are combined into a single dose form, such that a fixed amount of each active agent is administered with a single dose of the composition, for example, from a single actuation of the composition. “Aerosolizable” means that a composition can be in the form of an aerosol, such as when actuated through container.

Unless otherwise specified, the term “arformoterol” refers to arformoterol (N-[2-hydroxy-5-[(1R)-1-hydroxy-2-[[(2R)-1-(4-methoxyphenyl) propan-2-yl] amino] ethyl] phenyl] formamide) which has the structure:

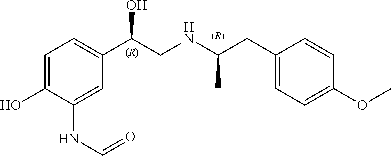

in any physical form, including any crystalline form (e.g., anhydrous, hydrate, or solvate form).

Unless otherwise specified, the term “glycopyrronium salt” refers to a pharmaceutical acceptable salt of glycopyrronium in any stereochemistry (e.g., S,S-, S,R-, R,S- or R,R-forms) or a mixture of such stereoisomers, e.g., a racemic mixture (S,S-, S,R-, R,S- and R,R-forms) or an enantiomerically enriched S,S-, S,R-, R,S- and R,R-forms of the pharmaceutical acceptable salt of glycopyrronium (i.e. pharmaceutically acceptable salt of (3S,2'S)-3-[(cyclopentylhydroxyphenylacetyl)oxy]-1, 1-dimethylpyrrolidinium, pharmaceutically acceptable salt of (3S,2′R)-3-[(cyclopentylhydroxyphenylacetyl)oxy]-1,1-dimethylpyrrolidinium, pharmaceutically acceptable salt of (3R,2'S)-3-[(cyclopentylhydroxyphenylacetyl)oxy]-1,1-dimethylpyrrolidinium and pharmaceutically acceptable salt of (3R,2′R)-3-[(cyclopentylhydroxyphenylacetyl)oxy]-1,1-dimethylpyrrolidinium).

“Salt” or “pharmaceutically acceptable salt” means those salts which are, within the scope of sound medical judgment, suitable for use in contact with the tissues of humans and lower animals without undue toxicity, irritation, and allergic response, commensurate with a reasonable benefit to risk ratio, and effective for their intended use. Representative salts include chloride, furoate, bromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, lauryl sulphate, sodium, calcium, potassium and magnesium.

“Effective amount” or “therapeutically effective amount” denotes an amount of an active ingredient that, when administered to a subject for treating a respiratory disorder, produces an intended therapeutic benefit in a subject.

“Active ingredient” (used interchangeably with “active” or “active agent” or “drug”) as used herein includes glycopyrronium and arformoterol and/or its salts.

The therapeutically effective amount of arformoterol or its salt to be administered per day may be about 0.001 μg to about 1000 μg, from about 0.01 μg to about 500 μg, more from about 0.1 μg to about 100 μg, about 0.2 μg to about 20 μg, or about 0.1 μg to about 7.5 μg. In certain embodiments, the discrete dosage strengths per actuation of arformoterol or its salt to be administered per day are 0.5 μg, 0.75 μg, 1 μg, 1.5 μg, 3.0 μg, 3.5 μg, 4.5 μg, 12 μg and 24 μg.

The therapeutically effective amount of glycopyrronium salt to be administered per day is about 0.1 μg to about 1000 μg, about 0.25 μg to about 100 μg, about 0.5 μg to about 80 μg, about 0.8 μg to about 50 μg, and or about 1 μg to about 30 μg. Preferably, the discrete dosage strengths of glycopyrronium salt to be administered per day are 3.5 μg, 3.75 μg, 7.5 μg, 11.25 μg, 12.5 μg, 14.4 μg, 15 μg, 25 μg, 30 μg, 44 μg and 50 μg.

A “stable” suspension is a suspension that does not undergo phase separation by visual observation. Stability is tested by shaking (e.g., for 1 minute or shaken once or twice) the composition, and a stable composition does not exhibit phase separation when stored for at least 5 minutes, 10 minutes, 20 minutes, 30 minutes, 2 hours, 6 hours, 12 hours or 24 hours at ambient conditions (e.g., about 25° C. and a relative humidity (RH) of about 60%) or at accelerated conditions (e.g., at about 40° C. and about 75% RH). In some embodiments, a “stable” aerosol is one in which the FPF or FPD do not change more than 15% or between 5-15% in 1 month from the initial FPF or FPD measured at ambient conditions (e.g., about 25° C. and a relative humidity (RH) of about 60%) or at accelerated conditions (e.g., at about 40° C. and about 75% RH). In some embodiments, a “stable” aerosol is one that does not contain a total impurity content of no more than 1.0% after storage at ambient conditions (at about 25° C. and a relative humidity of about 60%) for a period of at least 6 months.

“Treating” or “treatment” as used herein includes administration to alleviate symptoms and/or to prevent attacks of a disease, condition or disorder.

“Subject” includes mammals such as human and other animals, such as domestic animals (e.g., household pets including cats and dogs) and non-domestic animals (such as wildlife). Preferably, the subject is a human.

“Soluble” (used interchangeably with dissolved) means that a composition is either totally soluble in a particular solvent or it is sparingly soluble in that particular solvent, for example, a particular solute having a solubility of from 10 to 30 parts per solvent. The term soluble includes the definition of “very soluble” (less than 1 part of solvent per parts of solute), freely soluble (from 1 to 10 parts of solvent per part of solute), sparingly soluble (from 30 to 100 parts of solvent per part of solute) as given in Table 16-1 of Remington: The Science and Practice of Pharmacy, 20^{th} ed. Lippincott, Williams & Wilkins, 2001, p. 209, which is hereby incorporated by reference.

“Substantially insoluble” means that a composition is either totally insoluble in a particular solvent or it is poorly soluble in that particular solvent. The term “substantially insoluble” means that a particular solute has a solubility of less than one part per 100 parts solvent. The term “substantially insoluble” includes the definitions of “slightly soluble” (from 100 to 1000 parts solvent per 1 part solute), “very slightly soluble” (from 1000 to 10,000 parts solvent per 1 part solute) and “practically insoluble” (more than 10,000 parts solvent per 1 part solute) as given in Table 16-1 of Remington: The Science and Practice of Pharmacy, 21st ed. Lippincott, Williams & Wilkins, 2001, p. 209, which is hereby incorporated by reference.

“Pharmaceutically acceptable excipients” means any of the components of a pharmaceutical composition other than the active ingredients and which are approved by regulatory authorities or are generally regarded as safe for human or animal use.

“Average particle size” (or synonymously, “mean particle size”) refers to the distribution of particles, wherein about 50 volume percent of all the particles measured have a size less than the defined average particle size value and about 50 volume percent of all measurable particles measured have a particle size greater than the defined average particle size value. This can be identified by the term “D50” or “d (0.5)”. The average particle size can be measured using various techniques such as laser diffraction, photon correlation spectroscopy (PCS) and Coulter's principle.

Suspensions can include active agents in micronized form. Micronization typically involves milling and/or grinding to reduce the average diameter of active agent particles.

The following examples are provided to enable one skilled in the art to practice the invention and are merely illustrative of the invention. The examples should not be read as limiting the scope of the invention.

### EXAMPLES

### Abbreviations

The following abbreviations are used in the examples.

AT Arformoterol Tartrate

DCU Dose content uniformity

DUSA Dosage unit sampling apparatus

FPD Fine particle dose

FPF Fine particle fraction

GP Glycopyrronium bromide

GSD Geometric standard deviation

MDI Metered dose inhaler

MMAD Mass medium aerodynamic diameter

PVP Polyvinyl pyrrolidone, also called povidone

RH Relative humidity

### Example 1

### MDI Composition Comprising Arformoterol and Glycopyrronium Bromide

| | Qty. per | |
|---|---|---|
| | actuation in mg | |
| Ingredient | (Ex valve) | % w/w |
| Arformoterol Tartrate | 0.0034 | 0.0052 |
| Glycopyrronium Bromide | 0.0159 | 0.0245 |
| Polyethylene Glycol (PEG1000) | 0.0130 | 0.0200 |
| Povidone (PVP K25) | 0.000065 | 0.0001 |
| HFA227ea | 32.50 | 50.00 |
| HFA134a | q.s. to 65 mg | q.s. to 100 |

Manufacturing Process:

1. Povidone and Polyethylene Glycol 1000 were added to a manufacturing vessel.

2. Propellant HFA227ea was added to the manufacturing vessel of step 1 through a propellant addition valve and homogenized.

3. Arformoterol tartrate and Glycopyrronium bromide were added to the blend of step 2 and homogenized.

4. To the mixture obtained in step 3, Propellant HFA 134a was added into the manufacturing vessel through the propellant addition valve and homogenized.

5. The resulting homogenous suspension obtained in step 4 was filled into a suitable container.

Table 1 reports the stability data for glycopyrrolate and arformoterol tartrate, FPD, FPF, MMAD, DCU, and GSD at one month and two months. In this stability study and following stability studies % GP or AT is measured via HPLC. FPD, FPF, and MMAD are measured using an Anderson Cascade Impactor. DCU is measured using a DUSA apparatus.

TABLE 1
Stability Data

| | Example 1 | | | | | |
|---|---|---|---|---|---|---|
| | | | Results (1 M) | | Results (2 M) | |
| | Results (Initial) | | 40° C.-75% RH | | 40° C.-75% RH | |
| Test | GP | AT | GP | AT | GP | AT |
| Assay (%) | 97.12 | 97.99 | 98.20 | 99.00 | 102.00 | 101.24 |
| FPD | 6.30 | 1.45 | 6.06 | 1.24 | 6.29 | 1.38 |
| (mcg) | | | | | | |
| FPF (%) | 54.29 | 64.75 | 52.04 | 65.20 | 57.20 | 66.3 |
| MMAD | 2.6900 | 1.9800 | 3.1229 | 2.2500 | 2.8690 | 2.2454 |
| (μm) | | | | | | |
| GSD (μm) | 1.7400 | 1.8400 | 1.4418 | 1.6170 | 1.5905 | 1.6466 |

Table 2 reports the results of an assay measuring the percentage of each active ingredient in the initial, middle, and end dose of the aerosol from its container using HPLC.

TABLE 2
Assay values of different stages (Initial, Middle,
End Dose)

| | GP | | | AT | | |
|---|---|---|---|---|---|---|
| | Initial | 1 M | 2 M | Initial | 1 M | 2 M |
| Initial | 91.54 | 96.40 | 99.60 | 93.70 | 97.80 | 100.08 |
| Middle | 100.79 | 97.90 | 104.86 | 101.93 | 96.10 | 103.18 |
| End | 99.03 | 100.20 | 102.55 | 98.34 | 103.20 | 100.45 |
| Avg. | 97.12 | 98.20 | 102.34 | 97.99 | 99.00 | 101.24 |

### Example 2

### MDI Composition Comprising Arformoterol Tartrate and Glycopyrronium Bromide

| | Quantity per | | | |
|---|---|---|---|---|
| | actuation in mg | A1 | A2 | A3 |
| Ingredient | (ex- valve) | % w/w | % w/w | % w/w |
| Arformoterol Tartrate | 0.0052 | 0.0075 | | |
| Glycopyrronium | 0.0137 | 0.0196 | — | — |
| Bromide | 0.0091 | — | 0.0131 | — |
| | 0.0045 | — | — | 0.0065 |

| | | |
|---|---|---|
| Polyethylene Glycol (1000) | 0.0140 | 0.02 |
| Povidone (PVP K25) | 0.00007 | 0.0001 |
| HFA227ea | q.s to 70 | q.s to 100 |

Manufacturing Process:

1. Povidone and Polyethylene Glycol 1000 were added to a manufacturing vessel.

2. Propellant HFA227ea was added to the manufacturing vessel of step 1 through a propellant addition valve and homogenized.

3. Arformoterol tartrate was weighed and added into the vessel through a drug addition port.

4. Glycopyrronium bromide was weighed under isolator having RH below 15% and a temperature between 20-25° C. and added into the mixing vessel through a drug addition port.

5. Recirculation of Povidone and Polyethylene glycol 1000 and HFA 227ea solution was done through drug addition port under continuous stirring.

6. The resulting homogenous suspension obtained in step 5 was filled into a suitable container.

### Example 2A

### MDI Composition Comprising Arformoterol Tartrate and Glycopyrronium Bromide

| | Quantity per | | | |
|---|---|---|---|---|
| | actuation in mg | A1a | A2a | A3a |
| Ingredient | (ex- valve) | % w/w | % w/w | % w/w |
| Arformoterol Tartrate | 0.0053 | 0.0077 | | |
| Glycopyrronium | 0.0140 | 0.0201 | — | — |
| Bromide | 0.0093 | — | 0.0134 | — |
| | 0.0046 | — | — | 0.0067 |

| | | |
|---|---|---|
| Polyethylene Glycol (1000) | 0.0140 | 0.02 |
| Povidone (PVP K25) | 0.00007 | 0.0001 |
| HFA227ea | q.s to 70 | q.s to 100 |

Manufacturing Process:

The manufacturing process set forth in Example 2 was followed.

### Example 2B

### MDI Composition Comprising Arformoterol Tartrate and Glycopyrronium Bromide

| | Quantity per | | | |
|---|---|---|---|---|
| | actuation in mg | A1b | A2b | A3b |
| Ingredient | (ex-valve) | % w/w | % w/w | % w/w |
| Arformoterol Tartrate | 0.0053 | 0.0077 | | |
| Glycopyrronium | 0.0140 | 0.0201 | — | — |
| Bromide | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA134a | q.s to 70 | q.s to 100 |

Manufacturing Process:

1. Povidone and Polyethylene Glycol 1000 were added to a manufacturing vessel.

2. Propellant HFA134a was added to the manufacturing vessel of step 1 through a propellant addition valve and homogenized.

3. Arformoterol tartrate was weighed and added into the vessel through a drug addition port.

4. Glycopyrronium bromide was weighed under isolator having RH below 15% and a temperature between 20-25° C. and added into the mixing vessel through a drug addition port.

5. Recirculation of Povidone and Polyethylene glycol 1000 and HFA 134a solution was done through drug addition port under continuous stirring.

6. The resulting homogenous suspension obtained in step 5 was filled into a suitable container.

### Example 3

### MDI Composition Comprising Arformoterol and Glycopyrronium Bromide

| | Quantity per | | | |
|---|---|---|---|---|
| | actuation in mg | B1 | B2 | B3 |
| Ingredient | (ex-valve) | % w/w | % w/w | % w/w |
| Arformoterol Tartrate | 0.0026 | 0.0038 | | |
| Glycopyrronium | 0.0137 | 0.0196 | — | — |
| Bromide | 0.0091 | — | 0.0131 | — |
| | 0.0045 | — | — | 0.0065 |

| | | |
|---|---|---|
| Polyethylene Glycol | 0.0140 | 0.02 |
| (1000) | | |
| Povidone (PVP K25) | 0.00007 | 0.0001 |
| HFA227ea | q.s to 70 | q.s to 100 |

Manufacturing Process:

The manufacturing process set forth in Example 2 was followed.

The stability results (Initial, & 1 month) for arformoterol tartrate and glycopyrronium bromide are provided in Table 3.

TABLE 3
Stability Data

| Example 3 (B2) | |
|---|---|
| | Results (1 M) |
| | 40° C. - 75% |
| Results (Initial) | RH |

| Test | AT | GP | AT | GP |
|---|---|---|---|---|
| Assay (%) | 94.7 | 101.9 | 93.4 | 104 |
| FPD (mcg) | 0.97 | 4.45 | 0.98 | 4.43 |
| FPF (%) | 66.96 | 60.03 | 64.45 | 60.74 |
| MMAD (μm) | 2.13 | 2.44 | 2.10 | 2.35 |
| GSD (μm) | 1.65 | 1.71 | 1.65 | 1.71 |
| DCU (%) | 96.6 | 96.8 | 97.3 | 101.6 |

### Example 3A

### MDI Composition Comprising Arformoterol and Glycopyrronium Bromide

| | Quantity per | | | |
|---|---|---|---|---|
| | actuation in mg | B1a | B2a | B3a |
| Ingredient | (ex-valve) | % w/w | % w/w | % w/w |
| Arformoterol Tartrate | 0.0026 | 0.0038 | | |
| Glycopyrronium | 0.0140 | 0.0201 | — | — |
| Bromide | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA227ea | q.s to 70 | q.s to 100 |

Manufacturing Process:

The manufacturing process set forth in Example 2 was followed.

### Example 3B

### MDI Composition Comprising Arformoterol and Glycopyrronium Bromide

| | Quantity per | | | |
|---|---|---|---|---|
| | actuation in mg | B1b | B2b | B3b |
| Ingredient | (ex-valve) | % w/w | % w/w | % w/w |
| Arformoterol Tartrate | 0.0026 | 0.0038 | | |
| Glycopyrronium | 0.0140 | 0.0201 | — | — |
| Bromide | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA134a | q.s to 70 | q.s to 100 |

Manufacturing Process:

The manufacturing process set forth in Example 2B was followed.

### Example 4

### MDI Composition Comprising Arformoterol and Glycopyrronium Bromide

| | Quantity per | | | |
|---|---|---|---|---|
| | actuation in mg | C1 | C2 | C3 |
| Ingredient | (ex-valve) | % w/w | % w/w | % w/w |
| Arformoterol Tartrate | 0.0017 | 0.0025 | | |
| Glycopyrronium | 0.0137 | 0.0196 | — | — |
| Bromide | 0.0091 | — | 0.0131 | — |
| | 0.0045 | — | — | 0.0065 |

| | | |
|---|---|---|
| Polyethylene Glycol | 0.0140 | 0.02 |
| (1000) | | |
| Povidone (PVP K25) | 0.00007 | 0.0001 |
| HFA227ea | q.s to 70 | q.s |

Manufacturing Process:

The manufacturing process set forth in Example 2 was followed.

### Example 4A

### MDI Composition Comprising Arformoterol and Glycopyrronium Bromide

| | Quantity per | | | |
|---|---|---|---|---|
| | actuation in mg | C1a | C2a | C3a |
| Ingredient | (ex-valve) | % w/w | % w/w | % w/w |
| Arformoterol Tartrate | 0.0017 | 0.0026 | | |
| Glycopyrronium | 0.0140 | 0.0201 | — | — |
| Bromide | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA227ea | q.s to 70 | q.s |

Manufacturing Process:

The manufacturing process set forth in Example 2 was followed.

### Example 4B

### MDI Composition Comprising Arformoterol and Glycopyrronium Bromide

| | Quantity per | | | |
|---|---|---|---|---|
| | actuation in mg | C1b | C2b | C3b |
| Ingredient | (ex-valve) | % w/w | % w/w | % w/w |
| Arformoterol Tartrate | 0.0017 | 0.0026 | | |
| Glycopyrronium | 0.0140 | 0.0201 | — | — |
| Bromide | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA134a | q.s to 70 | q.s |

Manufacturing Process:

The manufacturing process set forth in Example 2b was followed.

### Example 5

### MDI Composition Comprising Arformoterol and Glycopyrronium Bromide

| | Quantity per | | | |
|---|---|---|---|---|
| | actuation in mg | D1 | D2 | D3 |
| Ingredient | (ex-valve) | % w/w | % w/w | % w/w |
| Arformoterol Tartrate | 0.0008 | 0.0013 | | |
| Glycopyrronium Bromide | 0.0137 | 0.0196 | — | — |
| | 0.0091 | — | 0.0131 | — |
| | 0.0045 | — | — | 0.0065 |

| | | |
|---|---|---|
| Polyethylene Glycol | 0.0140 | 0.02 |
| (1000) | | |
| Povidone (PVP K25) | 0.00007 | 0.0001 |
| HFA227ea | q.s to 70 | q.s to 100 |

Manufacturing Process:

The manufacturing process set forth in Example 2 was followed.

### Example 5A

### MDI Composition Comprising Arformoterol and Glycopyrronium Bromide

| | Quantity per | | | |
|---|---|---|---|---|
| | actuation in mg | D1a | D2a | D3a |
| Ingredient | (ex-valve) | % w/w | % w/w | % w/w |
| Arformoterol Tartrate | 0.0008 | 0.0013 | | |
| Glycopyrronium Bromide | 0.0140 | 0.0201 | — | — |
| | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA227ea | q.s to 70 | q.s to 100 |

Manufacturing Process:

The manufacturing process set forth in Example 2 was followed.

### Example 5B

### MDI Composition Comprising Arformoterol and Glycopyrronium Bromide

| | Quantity per | | | |
|---|---|---|---|---|
| | actuation in mg | D1b | D2b | D3b |
| Ingredient | (ex-valve) | % w/w | % w/w | % w/w |
| Arformoterol Tartrate | 0.0008 | 0.0013 | | |
| Glycopyrronium Bromide | 0.0140 | 0.0201 | — | — |
| | 0.0093 | — | 0.0134 | — |
| | 0.0046 | — | — | 0.0067 |

| | | |
|---|---|---|
| Polyethylene Glycol | 0.0140 | 0.02 |
| (1000) | | |
| Povidone (PVP K25) | 0.00007 | 0.0001 |
| HFA134a | q.s to 70 | q.s to 100 |

Manufacturing Process:

The manufacturing process set forth in Example 2b was followed.

### Example 6

### MDI Composition Comprising Glycopyrronium Bromide

| | Quantity per | | | |
|---|---|---|---|---|
| | actuation in | E1 | E2 | E3 |
| Ingredients | mg (ex-valve) | % w/w | | |
| Glycopyrronium Bromide | 0.0137 | 0.0196 | — | — |
| | 0.0091 | — | 0.0131 | — |
| | 0.0045 | — | — | 0.0065 |

| | | |
|---|---|---|
| Povidone K-25 | 0.00007 | 0.0001 |
| Polyethylene Glycol 1000 | 0.0140 | 0.02 |
| HFA 227 ea | q.s. to 70 | q.s. to 100 |

Manufacturing Process:

1. Povidone and Polyethylene Glycol 1000 were added to a manufacturing vessel.

2. Propellant HFA227ea was added to the manufacturing vessel of step 1 through a propellant addition valve and homogenized.

3. Glycopyrronium bromide was weighed under isolator having RH below 15% and a temperature between 20-25° C. and added into the mixing vessel through a drug addition port.

4. Recirculation of Povidone and Polyethylene glycol 1000 and HFA 227ea solution was done through a drug addition port under continuous stirring.

5. The resulting homogenous suspension obtained in step 5 was filled into a suitable container.

The stability results (Initial & 1 month) for glycopyrronium bromide are provided in Table 4.

TABLE 4
Stability Data

Example 6 (E2)

| | | Results (1 M) |
|---|---|---|
| Test | (Initial) | 40° C. - 75% RH |
| Assay ⁽%) | 95.7 | 97.2 |
| FPD (mcg) | 4.88 | 4.65 |
| FPF (%) | 63.56 | 62.12 |
| MMAD (μm) | 2.21 | 2.30 |
| GSD (μm) | 1.79 | 1.75 |
| DCU (%) | 105.78 | 98.01 |

### Example 6A

### MDI Composition Comprising Glycopyrronium Bromide

| | Quantity per | | | |
|---|---|---|---|---|
| | actuation in | E1a | E2a | E3a |
| Ingredients | mg (ex-valve) | % w/w | | |
| Glycopyrronium Bromide | 0.0140 | 0.0201 | — | — |
| | 0.0093 | — | 0.0134 | — |
| | 0.0046 | — | — | 0.0067 |

| | | |
|---|---|---|
| Povidone K-25 | 0.00007 | 0.0001 |
| Polyethylene Glycol 1000 | 0.0140 | 0.02 |
| HFA 227 ea | q.s. to 70 | q.s. to 100 |

Manufacturing Process:

The manufacturing process set forth in Example 6 was followed.

### Example 6B

### MDI Composition Comprising Glycopyrronium Bromide

| | Quantity per | | | |
|---|---|---|---|---|
| | actuation in | E1b | E2b | E3b |
| Ingredients | mg (ex-valve) | % w/w | | |
| Glycopyrronium Bromide | 0.0140 | 0.0201 | — | — |
| | 0.0093 | — | 0.0134 | — |
| | 0.0046 | — | — | 0.0067 |

| | | |
|---|---|---|
| Povidone K-25 | 0.00007 | 0.0001 |
| Polyethylene Glycol 1000 | 0.0140 | 0.02 |
| HFA 134a | q.s. to 70 | q.s. to 100 |

Manufacturing Process:

1. Povidone and Polyethylene Glycol 1000 were added to a manufacturing vessel.

2. Propellant HFA134a was added to the manufacturing vessel of step 1 through a propellant addition valve and homogenized.

3. Glycopyrronium bromide was weighed under isolator having RH below 15% and a temperature between 20-25° C. and added into the mixing vessel through a drug addition port.

4. Recirculation of Povidone and Polyethylene glycol 1000 and HFA 134a solution was done through a drug addition port under continuous stirring.

5. The resulting homogenous suspension obtained in step 5 was filled into a suitable container.

### Example 7

### MDI Composition Comprising Arformoterol Tartrate

| | Quantity per | | | | |
|---|---|---|---|---|---|
| | actuation in mg | F1 | F2 | F3 | F4 |
| Ingredients | (ex-valve) | % w/w | | | |
| Arformoterol | 0.0052 | 0.0075 | — | — | — |
| Tartrate | 0.0026 | — | 0.0038 | — | — |
| | 0.0017 | — | — | 0.0025 | — |
| | 0.0008 | — | — | — | 0.0013 |

| | | |
|---|---|---|
| Povidone K-25 | 0.00007 | 0.0001 |
| Polyethylene | 0.0140 | 0.02 |
| Glycol 1000 | | |
| HFA 227 ea | q.s. to 70 | q.s. to 100 |

Manufacturing Process:

1. Povidone and Polyethylene Glycol 1000 were added to a manufacturing vessel.

2. Propellant HFA227ea was added to the manufacturing vessel of step 1 through a propellant addition valve and homogenized.

3. Arformoterol Tartrate was weighed under isolator having RH below 15% and a temperature between 20-25° C. and added into the mixing vessel through a drug addition port.

4. Recirculation of Povidone and Polyethylene glycol 1000 and HFA 227ea solution was done through a drug addition port under continuous stirring.

5. The resulting homogenous suspension obtained in step 5 was filled into a suitable container.

The stability results (Initial & 3 months) for arformoterol tartrate are provided in Table 5.

TABLE 5
Stability Data

Example 7 (F2)

| | | Results (3M) |
|---|---|---|
| Test | (Initial) | 40° C.-75% RH |
| Assay (%) | 93.4 | 99.55 |
| FPD (mcg) | 0.9005 | 0.9312 |
| FPF (%) | 70.19 | 57.41 |
| MMAD (μm) | 2.3053 | 2.4495 |
| GSD(μm) | 1.5791 | 1.6832 |
| DCU (%) | 102.49 | 103.50 |

### Example 7A

### MDI Composition Comprising Arformoterol Tartrate

| | Quantity per | | | | |
|---|---|---|---|---|---|
| | actuation in mg | F1a | F2a | F3a | F4a |
| Ingredients | (ex-valve) | % w/w | | | |
| Arformoterol | 0.0053 | 0.0077 | — | — | — |
| Tartrate | 0.0026 | — | 0.0038 | — | — |
| | 0.0017 | — | — | 0.0026 | — |
| | 0.0008 | — | — | — | 0.0013 |

| | | |
|---|---|---|
| Povidone K-25 | 0.00007 | 0.0001 |
| Polyethylene | 0.0140 | 0.02 |
| Glycol 1000 | | |
| HFA 227 ea | q.s. to 70 | q.s. to 100 |

Manufacturing Process:

The manufacturing process set forth in Example 7 was followed.

### Example 7B

### MDI Composition Comprising Arformoterol Tartrate

| | Quantity per | | | | |
|---|---|---|---|---|---|
| | actuation in mg | F1b | F2b | F3b | F4b |
| Ingredients | (ex-valve) | % w/w | | | |
| Arformoterol | 0.0053 | 0.0077 | — | — | — |
| Tartrate | 0.0026 | — | 0.0038 | — | — |
| | 0.0017 | — | — | 0.0026 | — |
| | 0.0008 | — | — | — | 0.0013 |

| | | |
|---|---|---|
| Povidone K-25 | 0.00007 | 0.0001 |
| Polyethylene | 0.0140 | 0.02 |
| Glycol 1000 | | |
| HFA 134a | q.s. to 70 | q.s. to 100 |

Manufacturing Process:

1. Povidone and Polyethylene Glycol 1000 were added to a manufacturing vessel.

2. Propellant HFA134a was added to the manufacturing vessel of step 1 through a propellant addition valve and homogenized.

3. Arformoterol Tartrate was weighed under isolator having RH below 15% and a temperature between 20-25° C. and added into the mixing vessel through a drug addition port.

4. Recirculation of Povidone and Polyethylene glycol 1000 and HFA 134a solution was done through a drug addition port under continuous stirring.

5. The resulting homogenous suspension obtained in step 5 was filled into a suitable container.

### Examples 8-13

### MDI Compositions Containing Arformoterol Tartrate

| | Composition | | | | | |
|---|---|---|---|---|---|---|
| | (% w/w) | | | | | |
| Ingredient | Exp. 8 | Exp. 9 | Exp. 10 | Exp. 11 | Exp. 12 | Exp.13 |
| Arformoterol | 0.00122 | 0.00181 | 0.00242 | 0.00363 | 0.00496 | 0.00726 |
| Tartrate | | | | | | |
| Povidone K25 | 0.0001 | 0.0001 | 0.0001 | 0.0001 | 0.0001 | 0.0001 |
| Polyethylene | 0.02 | 0.02 | 0.02 | 0.02 | 0.02 | 0.02 |
| Glycol 1000 | | | | | | |
| Propellant HFA227 | q.s to 100 | q.s to 100 | q.s to 100 | q.s to 100 | q.s to 100 | q.s to 100 |

Manufacturing Process:

1. Povidone K25 and Polyethylene Glycol 1000 are added to a manufacturing vessel.

2. Part quantity of Propellant HFA227 is added into the manufacturing vessel through a propellant addition valve and homogenized.

3. Arformoterol Tartrate is added to the blend of step-2 and homogenized.

4. The blend from step-3 is mixed homogenously with the remaining quantity of Propellant HFA 227.

5. The resulting homogenous suspension obtained in step 4 was filled into a suitable container.

TABLE 6
Stability Study of Example 12 Composition

| | Parameters for Arformoterol Tartrate | | | |
|---|---|---|---|---|
| | | 1 Month | 2 Month | 3 Month |
| Test | Initial | (40° C.-75% RH) | (40° C.-75% RH) | (40° C.-75% RH) |
| FPD (mcg) | 1.34 | 1.56 | 1.20 | 1.36 |
| FPF (%) | 59 | 65 | 50 | 55 |
| MMAD (μm) | 2.60 | 2.10 | 2.50 | 2.90 |
| GSD | 1.60 | 1.69 | 1.66 | 1.56 |
| Assay (%) | 101.9 | 99.2 | 100.5 | 99.2 |
| By HPLC | | | | |

Spray Pattern Analysis by Spray view (Proveris) Distance: 60 mm

| | | | | |
|---|---|---|---|---|
| Major Axis (mm) | 16 | 16 | 21 | 21 |
| Minor Axis (mm) | 15 | 15 | 20 | 20 |
| Ovality Ratio | 1.12 | 1.13 | 1.09 | 1.11 |
| Pattern Area (mm²) | 191 | 195 | 335 | 330 |

Uniformity of Delivered Dose (%)

| | | | | |
|---|---|---|---|---|
| Min | 104 | 95 | 94 | 97 |
| Max | 108 | 101 | 97 | 99 |
| Beginning | 106 | 97 | 96 | 99 |
| Middle | 107 | 99 | 95 | 98 |
| End | 107 | 96 | 95 | 98 |

Thus, the composition of Example 12 was found to be stable for three months at accelerated stability conditions (40° C.-75% RH) for arformoterol tartrate related parameters.

### Example 14

### MDI Composition Comprising Glycopyrronium Bromide and Arformoterol Tartrate

| | Qty. per actuation in | |
|---|---|---|
| Ingredient | mg (Ex valve) | % w/w |
| Arformoterol Tartrate | 0.0023 | 0.0048 |
| Glycopyrronium Bromide | 0.0126 | 0.0181 |
| Polyethylene Glycol (PEG1000) | 0.0140 | 0.02 |
| Povidone (PVP K25) | 0.00007 | 0.0001 |
| HFA227ea | Q.s to 70 | Q.s. to 100 |

Manufacturing Process:

The manufacturing process set forth in Example 2 was followed.

TABLE 7
Comparative data on Dispensing of API at controlled
RH and Normal RH

| At Normal RH | Controlled RH |
|---|---|
| (35-45%) | (Below 15%) |

| Test | AT | GB | AT | GB |
|---|---|---|---|---|
| Assay (%) | 107.18 | 92.81 | 97.99 | 97.12 |
| DCU (%) | 85.36 | 70.39 | 106.00 | 93.01 |

### Example 15

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0045 | 0.0026 |
| Glycopyrrolate (Micronized) | 0.0156 | 0.0093 |
| HFA134a | 99.98 | 59.99 |
| Total | 100.0 | 60.00 |

Manufacturing Process:

1. Propellant HFA 134a was added to the manufacturing vessel.

2. Arformoterol tartrate was weighed and added into the vessel through a drug addition port.

3. Glycopyrronium bromide was weighed under isolator having RH below 15% and a temperature between 20-25° C. and added into the mixing vessel through a drug addition port.

4. Recirculation of Propellant HFA 134a was done through drug addition port under continuous stirring.

5. The resulting homogenous suspension obtained in step 4 was filled into a suitable container.

### Example 16

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0038 | 0.0026 |
| Glycopyrrolate (Micronized) | 0.0134 | 0.0093 |
| HFA227ea | 99.98 | 69.99 |
| Total | 100.0 | 70.00 |

Manufacturing Process:

1. Propellant HFA 227ea was added to the manufacturing vessel.

2. Arformoterol tartrate was weighed and added into the vessel through a drug addition port.

3. Glycopyrronium bromide was weighed under isolator having RH below 15% and a temperature between 20-25° C. and added into the mixing vessel through a drug addition port.

4. Recirculation of Propellant HFA 227ea was done through drug addition port under continuous stirring.

5. The resulting homogenous suspension obtained in step 4 was filled into a suitable container.

### Example 17

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0038 | 0.0026 |
| Glycopyrrolate (Micronized) | 0.0134 | 0.0093 |
| Polyethylene Glycol (1000) | 0.0200 | 0.0140 |
| HFA227ea | 99.96 | 69.97 |
| Total | 100.0 | 70.00 |

Manufacturing Process:

1. Polyethylene Glycol 1000 was added to a manufacturing vessel.

2. Propellant HFA 227ea was added to the manufacturing vessel of step 1 through a propellant addition valve and homogenized.

3. Arformoterol tartrate was weighed and added into the vessel through a drug addition port.

4. Glycopyrronium bromide was weighed under isolator having RH below 15% and a temperature between 20-25° C. and added into the mixing vessel through a drug addition port.

5. Recirculation of Polyethylene glycol 1000 and Propellant HFA 227ea solution was done through drug addition port under continuous stirring.

6. The resulting homogenous suspension obtained in step 5 was filled into a suitable container.

### Example 18

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0038 | 0.0026 |
| Glycopyrrolate (Micronized) | 0.0134 | 0.0093 |
| Povidone (PVP K25) | 0.0001 | 0.00007 |
| HFA227ea | 99.98 | 69.99 |
| Total | 100.0 | 70.00 |

Manufacturing Process:

1. Povidone was added to a manufacturing vessel.

2. Propellant HFA 227ea was added to the manufacturing vessel of step 1 through a propellant addition valve and homogenized.

3. Arformoterol tartrate was weighed and added into the vessel through a drug addition port.

4. Glycopyrronium bromide was weighed under isolator having RH below 15% and a temperature between 20-25° C. and added into the mixing vessel through a drug addition port.

5. Recirculation of Povidone and Propellant HFA 227ea solution was done through drug addition port under continuous stirring.

6. The resulting homogenous suspension obtained in step 5 was filled into a suitable container.

### Example 19

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0040 | 0.0026 |
| Glycopyrrolate (Micronized) | 0.0138 | 0.0093 |
| Polyethylene Glycol (1000) | 0.0200 | 0.0136 |
| Povidone (PVP K25) | 0.0001 | 0.000068 |
| HFA134a | 20.00 | 13.60 |
| HFA227ea | 79.96 | 54.37 |
| Total | 100.0 | 68.00 |

Manufacturing Process:

1. Povidone and Polyethylene Glycol 1000 were added to a manufacturing vessel.

2. Propellant HFA134a & HFA 227ea were added to the manufacturing vessel of step 1 through a propellant addition valve and homogenized.

3. Arformoterol tartrate was weighed and added into the vessel through a drug addition port.

4. Glycopyrronium bromide was weighed under isolator having RH below 15% and a temperature between 20-25° C. and added into the mixing vessel through a drug addition port.

5. Recirculation of Povidone and Polyethylene glycol 1000 and Propellant HFA134a & HFA 227ea solution were done through drug addition port under continuous stirring.

6. The resulting homogenous suspension obtained in step 5 was filled into a suitable container.

### Example 20

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0041 | 0.0026 |
| Glycopyrrolate (Micronized) | 0.0142 | 0.0093 |
| Polyethylene Glycol (1000) | 0.0200 | 0.0132 |
| Povidone (PVP K25) | 0.0001 | 0.000066 |
| HFA134a | 40.00 | 26.40 |
| HFA227ea | 59.96 | 39.57 |
| Total | 100.0 | 66.00 |

Manufacturing Process:

The manufacturing process set forth in Example 19 was followed.

### Example 21

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0042 | 0.0026 |
| Glycopyrrolate (Micronized) | 0.0146 | 0.0093 |
| Polyethylene Glycol (1000) | 0.0200 | 0.0128 |
| Povidone (PVP K25) | 0.0001 | 0.000064 |
| HFA134a | 60.00 | 38.40 |
| HFA227ea | 39.96 | 25.58 |
| TOTAL | 100.0 | 64.00 |

Manufacturing Process:

The manufacturing process set forth in Example 19 was followed.

### Example 22

### MDI Composition Comprising Arformoterol and Glycopyrronium Bromide

| | | Qty/Actuation (mg) |
|---|---|---|
| Ingredient | % w/w | (ex-valve) |
| Arformoterol Tartrate (Micronized) | 0.0043 | 0.0026 |
| Glycopyrrolate (Micronized) | 0.0149 | 0.0093 |
| Polyethylene Glycol (1000) | 0.0200 | 0.0126 |
| Povidone (PVP K25) | 0.0001 | 0.000063 |
| HFA134a | 70.00 | 44.10 |
| HFA227ea | 29.96 | 18.88 |
| Total | 100.0 | 63.00 |

Manufacturing Process:

The manufacturing process set forth in Example 19 was followed.

### Example 23

### Additional MDI Compositions Comprising Glycopyrronium Bromide and Arformoterol Tartrate

The disclosure includes the following MDI compositions, which can be prepared by the methods disclosed in Examples 1-22. Gly=Glycopyrronium bromide, Arf=arformoterol tartrate (weight of free base given), Act=Actuation. Propellant HFA 227ea or HFA 134 or mixture of HFA 227ea and HFA 134 is added q.s.

TABLE 8
Additional Exemplary Compositions

| | | | | | | PVPK25 | PEG1000 |
|---|---|---|---|---|---|---|---|
| Strength (μg) | | Gly | | Arf | | (% w/w, | (% w/w, |
| Gly | Arf | (% w/w, μg/act) | | (% w/w, μg/act) | | μg/act) | μg/act) |
| 11.25 | 3 | 0.0196/ | 0.0201/ | 0.0052/ | 0.0054/ | 0.0001/ | 0.02/14.000 |
| | | 13.725 | 14.0625 | 3.660 | 3.7500 | 0.070 | |
| 7.5 | 3 | 0.0131/ | 0.0134/ | 0.0052/ | 0.0054/ | 0.0001/ | 0.02/14.000 |
| | | 9.15 | 9.3750 | 3.660 | 3.7500 | 0.070 | |
| 3.75 | 3 | 0.0065/ | 0.0067/ | 0.0052/ | 0.0054/ | 0.0001/ | 0.02/14.000 |
| | | 4.575 | 4.6875 | 3.660 | 3.7500 | 0.070 | |
| 11.25 | 1.5 | 0.0196/ | 0.0201/ | 0.0026/ | 0.0027/ | 0.0001/ | 0.02/14.000 |
| | | 13.725 | 14.0625 | 1.830 | 1.8750 | 0.070 | |
| 7.5 | 1.5 | 0.0131/ | 0.0134/ | 0.0026/ | 0.0027/ | 0.0001/ | 0.02/14.000 |
| | | 9.15 | 9.3750 | 1.830 | 1.8750 | 0.070 | |
| 3.75 | 1.5 | 0.0065/ | 0.0067/ | 0.0026/ | 0.0027/ | 0.0001/ | 0.02/14.000 |
| | | 4.575 | 4.6875 | 1.830 | 1.8750 | 0.070 | |
| 11.25 | 1 | 0.0196/ | 0.0201/ | 0.0017/ | 0.0018/ | 0.0001/ | 0.02/14.000 |
| | | 13.725 | 14.0625 | 1.220 | 1.2500 | 0.070 | |
| 7.5 | 1 | 0.0131/ | 0.0134/ | 0.0017/ | 0.0018/ | 0.0001/ | 0.02/14.000 |
| | | 9.15 | 9.3750 | 1.220 | 1.2500 | 0.070 | |
| 3.75 | 1 | 0.0065/ | 0.0067/ | 0.0017/ | 0.0018/ | 0.0001/ | 0.02/14.000 |
| | | 4.575 | 4.6875 | 1.220 | 1.2500 | 0.070 | |
| 11.25 | 0.5 | 0.0196/ | 0.0201/ | 0.0009/ | 0.0009/ | 0.0001/ | 0.02/14.000 |
| | | 13.725 | 14.0625 | 0.610 | 0.6250 | 0.070 | |
| 7.5 | 0.5 | 0.0131/ | 0.0134/ | 0.0009/ | 0.0009/ | 0.0001/ | 0.02/14.000 |
| | | 9.15 | 9.3750 | 0.610 | 0.6250 | 0.070 | |
| 3.75 | 0.5 | 0.0065/ | 0.0067/ | 0.0009/ | 0.0009/ | 0.0001/ | 0.02/14.000 |
| | | 4.575 | 4.6875 | 0.610 | 0.6250 | 0.070 | |

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A fixed dose pharmaceutical composition, comprising about 0.0005% w/w to about 0.05% w/w of arformoterol or its salt and about 0.005% w/w to about 0.05% w/w of a glycopyrronium salt, based upon the total weight of the composition, wherein the composition is in the form of an aerosol or an aerosolizable composition for inhalation administration.

2. The fixed dose pharmaceutical composition of claim 1, wherein the arformoterol or its salt and the glycopyrronium salt are present in a weight ratio of arformoterol or its salt to glycopyrronium salt of about 1:0.001 to about 1:100.

3. The fixed dose pharmaceutical composition of claim 1, wherein the arformoterol salt is arformoterol tartrate and the glycopyrronium salt is glycopyrronium bromide.

4. The fixed dose pharmaceutical composition of claim 1, wherein the arformoterol or its salt, the glycopyrronium salt, or both, are suspended in particulate form in the composition.

5. The fixed dose pharmaceutical composition of claim 1, further comprising polyvinyl pyrrolidone, polyethylene glycol, a propellant, or a combination thereof.

6. The fixed dose pharmaceutical composition of claim 5, wherein the composition comprises the polyvinyl pyrrolidone in substantially insoluble form and the polyethylene glycol in dissolved form.

7. The fixed dose pharmaceutical composition of claim 5, wherein the propellant is HFA 134a, HFA 227, or a mixture of HFA 227 and HFA 134a.

8. The fixed dose pharmaceutical composition of claim 7, wherein the composition comprises at least about 30% w/w of the HFA 227, or at least about 30% w/w of the HFA 134a in the mixture of HFA 227 and HFA 134a, based upon the weight of the propellant.

9. The fixed dose pharmaceutical composition of claim 5, wherein the composition comprises about 0.00001% w/w to about 0.01% w/w of the polyvinyl pyrrolidone, based upon the total weight of the fixed dose pharmaceutical composition.

10. The fixed dose pharmaceutical composition of claim 9, wherein the composition comprises less than about 0.0015% w/w polyvinyl pyrrolidone, based upon the total weight of the fixed dose pharmaceutical composition.

11. The fixed dose pharmaceutical composition of claim 5, wherein the composition comprises about 0.005% w/w to about 0.08% w/w of the polyethylene glycol, based upon the total weight of the fixed dose pharmaceutical composition.

12. The fixed dose pharmaceutical composition of claim 5, wherein the composition is stable and does not undergo phase separation when stored at 25±2° C. and 60%±5% relative humidity for at least 30 minutes.

13. A fixed dose pharmaceutical suspension in the form of an aerosol or an aerosolizable composition for inhalation administration comprising (a) about 0.0005% w/w to about 0.05% w/w of arformoterol or its salt (b) about 0.005% w/w to about 0.05% w/w of a glycopyrronium salt (c) about 0.02% w/w of polyethylene glycol 1000, (d) about 0.0001% w/w of polyvinyl pyrrolidone K25 (PVP K25), and (e) propellant selected from HFA 134a, HFA 227, or a mixture of HFA 227 and HFA 134a, all weights based on the total weight of the composition.

14. The fixed dose pharmaceutical suspension of claim 13, wherein the arformoterol salt is arformoterol tartrate and the glycopyrronium salt is glycopyrronium bromide.

15. The fixed dose pharmaceutical composition of claim 13, wherein the total weight of the arformoterol or its salt and/or the total weight of the glycopyrronium salt is equal to or more than the total weight of the polyvinyl pyrrolidone.

16. The fixed dose pharmaceutical composition of claim 13, wherein the weight of the arformoterol or its salt or the weight the glycopyrronium salt is equal to or less than the total weight of the polyethylene glycol.

17. The fixed dose pharmaceutical composition of claim 13, wherein the composition is free or substantially free of an organic acid, an inorganic acid, or both.

18. A fixed dose pharmaceutical suspension in the form of an aerosol or an aerosolizable composition for inhalation administration comprising (a) an arformoterol salt, equivalent to about 0.1 μg to about 7.5 μg of arformoterol per actuation (b) 1 μg to about 30 μg per actuation of glycopyrronium salt, based on a weight of glycopyrronium bromide (c) about 0.02% w/w of polyethylene glycol 1000, (d) about 0.0001% w/w of polyvinyl pyrrolidone K25 (PVP K25), and (e) HFA 227, all weights based on the total weight of the composition.

19. The fixed dose pharmaceutical suspension of claim 18, wherein the aerosol composition exhibits a fine particle fraction of about 40% to about 80%.

20. The fixed dose pharmaceutical suspension of claim 18, wherein the aerosol composition exhibits a Mass Median Aerodynamic Diameter (MMAD) of about 1 μm to about 5 μm.

21. A method of treating a respiratory disorder in a subject in need thereof, comprising administering by inhalation the composition of claim 1 to the subject.
